(19) United States
(12) Patent Application Publication
(10) Pub. No.: US 2024/0409613 A1
(43) Pub. Date: Dec. 12, 2024

(54) Hemoglobin-Based Oxygen Carrier and Method of Preparation

(71) Applicant: The Governing Council of the University of Toronto, Toronto (CA)

(72) Inventors: Nathan WU, Toronto (CA); Ronald KLUGER, North York (CA)

(73) Assignee: The Governing Council of the University of Toronto, Toronto, ON (CA)

(21) Appl. No.: 18/737,128

(22) Filed: Jun. 7, 2024

Related U.S. Application Data
(60) Provisional application No. 63/507,028, filed on Jun. 8, 2023.

(30) Foreign Application Priority Data
Jun. 9, 2023 (CA) 3202703

(51) Int. Cl. C07K 14/805; A61K 38/00; C07F 9/09

(52) U.S. Cl. CPC C07K 14/805; C07F 9/096; A61K 38/00

(57) ABSTRACT

An azide-containing cross-linker and method of preparing the same are provided. Such cross-linker can be used as an intermediate in a scalable production of a cross-linked hemoglobin protein having its beta subunits covalently linked to the azide-containing cross-linker. The present disclosure also pertains to a hemoglobin-based oxygen carrier (HBOC) comprising a pair of azide cross-linked hemoglobin proteins coupled by a copper (I) catalyzed azide-alkyne cycloaddition (CuAAC) and a method for preparing the same. The HBOC may be used in a method for increasing oxygen transport or for use in transfusions or perfusions.

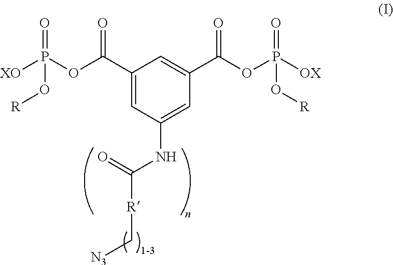

### CROSS-REFERENCE TO RELATED APPLICATIONS

This application claims priority to U.S. Provisional Patent Application No. 63/507,028 filed on Jun. 8, 2023 and to Canadian Patent Application No. 3,202,703 filed on Jun. 9, 2023, the entire contents of which are incorporated herein by reference.

### FIELD OF THE INVENTION

The present disclosure relates to a cross-linker for use in the preparation of a cross-linked hemoglobin protein and in particular to an azide-containing cross-linker as an intermediate in a scalable production of the cross-linked hemoglobin protein. The present disclosure also pertains to a cross-linked hemoglobin protein having its beta (β) subunits covalently linked to the azide-containing cross-linker, a hemoglobin-based oxygen carrier (HBOC) comprising a pair of such cross-linked hemoglobin proteins that are coupled by a copper (I) catalyzed azide-alkyne cycloaddition (CuAAC) and a method for preparing the HBOC. The HBOC may be used in a method for increasing oxygen transport or for use in transfusions or perfusions.

### BACKGROUND

Red cells from whole blood are routinely used in transfusion medicine. Their utility can be limited by compatibility of blood types, shelf life, required refrigeration as well as the possibility of undetected viral or bacterial contamination. These limitations led to the development of potential alternatives to red cells including sterile acellular HBOCs.

Hemoglobin is the oxygen-carrying component of red cells. Human adult hemoglobin protein is a tetramer having a molecular weight of approximately 64 kD and comprised of two alpha (α) and two beta (β) subunits. While hemoglobin is an effective oxygen carrier within the red cells, the tetrameric (αβ)₂ protein spontaneously dissociates into non-functional αβ dimers in circulation outside a cell. Chemical cross-linking between hemoglobin's constituent αβ-dimers may prevent such dissociation, while retaining the protein's oxygen transport. Clinical trials of early examples of cross-linked HBOCs revealed serious side effects, typically associated with vasoconstriction leading to hypertension. It was postulated that this was likely to be the result of circulating cross-linked hemoglobins penetrating endothelia, where the hemes within the HBOCs readily bind locally endogenous nitric oxide (NO), an endothelial relaxation factor.

It was postulated that HBOCs about twice the size of the hemoglobin tetramer are likely to be too large to traverse endothelia and would thereby carry oxygen in circulation without inducing hypertension.

Early processes for preparing oxygen-carrying bis-tetramers derived from hemoglobin appeared not to be sufficiently efficient to permit scale-up for potential clinical use. The final products would require extensive purification to avoid contamination by smaller assemblies that would be vasoactive, inducing clinical complications. For example, a cross-linker used in an early process to prepare the cross-linked hemoglobin did not discriminate between the two α and two β subunits, although only hemoglobins cross-linked at their β subunits can be coupled to produce the hemoglobin bis-tetramers. Consequently, the overall yield of the bis-tetramer would be low, and the inefficient process could not be scalable. Therefore, these early processes would not be adaptable for clinical perfusion or transfusion applications.

Thus, in one aspect, it would be desirable to provide a scalable process for producing HBOCs comprising cross-linked hemoglobin bis-tetramers.

In another aspect, it would be desirable to provide novel cross-linkers that can selectively bind to the amino groups of lysines (preferably position 82) of the β subunits of hemoglobin such as adult human hemoglobin (HbA), preferably with selectivity to avoid cross-linking reactions between the amino groups in the a subunits.

It is desirable that the HBOCs prepared using the novel cross-linkers provided herein may be used to increase oxygen transport and may thus have applications in transfusions or perfusions.

### SUMMARY

It has been found that the efficiency and scalability of the formation of β-β cross-linked hemoglobin is improved when using novel azide-containing cross-linkers, wherein the azide moiety is farther from the protein than in previously reported entities, while introducing the cross-linking in the desired site within the β subunits.

In one aspect, the present disclosure provides an azide-containing cross-linker, wherein the cross-linker is a compound of Formula I, II, III or IV:

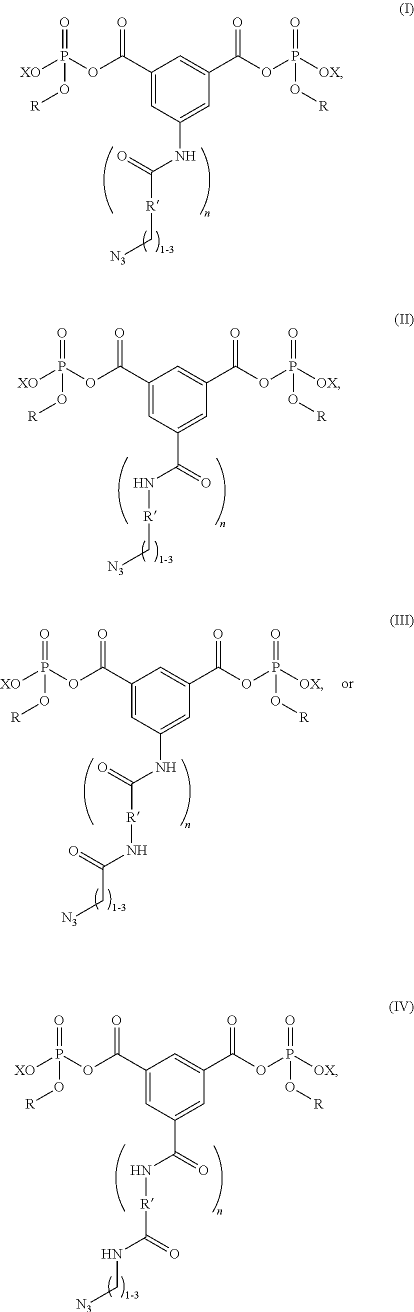

wherein R is an alkyl;
R′ is a para- or meta-substitutable benzene or benzene isomer, a para- or meta-substitutable heterocyclic six-membered aromatic ring or an isomer thereof, a five-membered aromatic ring or an isomer thereof, an indole ring, an indole derivative, a naphthalene or a naphthalene derivative, wherein each of the para- or meta-substitutable benzene, the para- or meta-substitutable benzene isomer, the para- or meta-substitutable heterocyclic six-membered aromatic ring or the isomer thereof, the five-membered aromatic ring or the isomer thereof, the indole ring, the indole derivative, the naphthalene and the naphthalene derivative is optionally substituted;
X is an alkali ion; and
n is 1 to 5.

In another aspect, an azide-containing cross-linker of Formula V is provided:

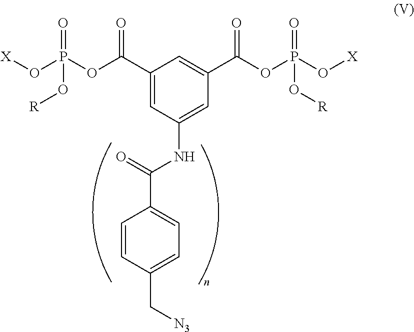

wherein R is an alkyl; 4
X is an alkali ion; and
n is 1 to 5.

In a further aspect, the following azide-containing cross-linker

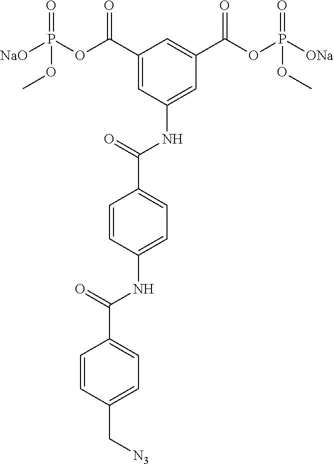

is provided.

In yet another aspect, there is provided a method for preparing an azide-containing cross-linker, wherein the method is one of the following reaction routes depending on the azide-containing cross-linker to be prepared:

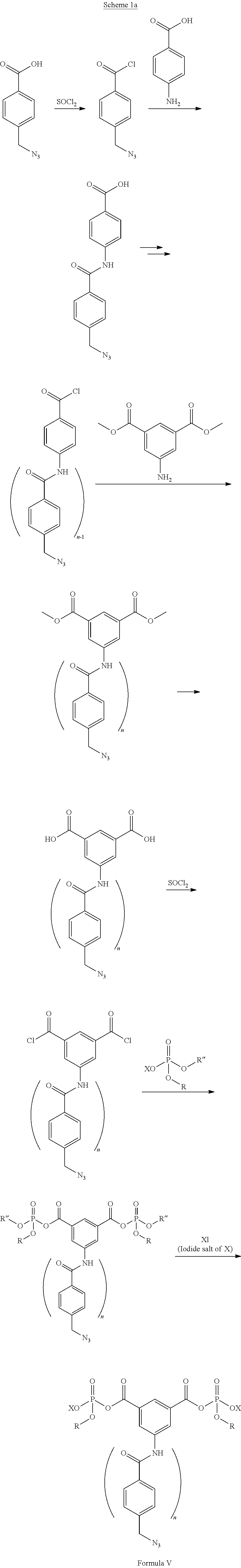

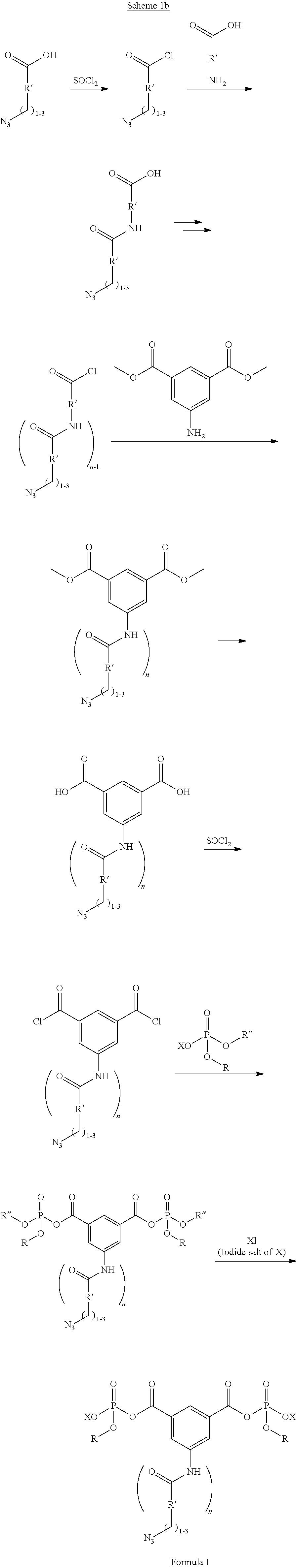

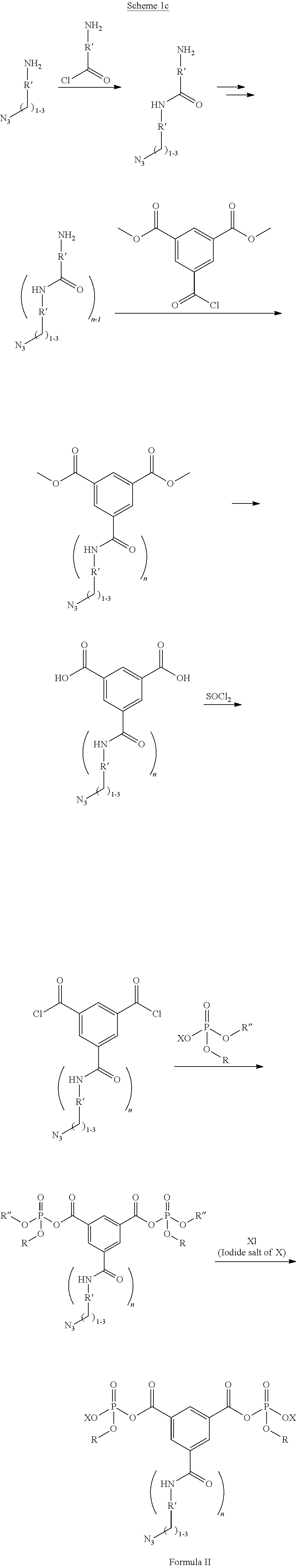

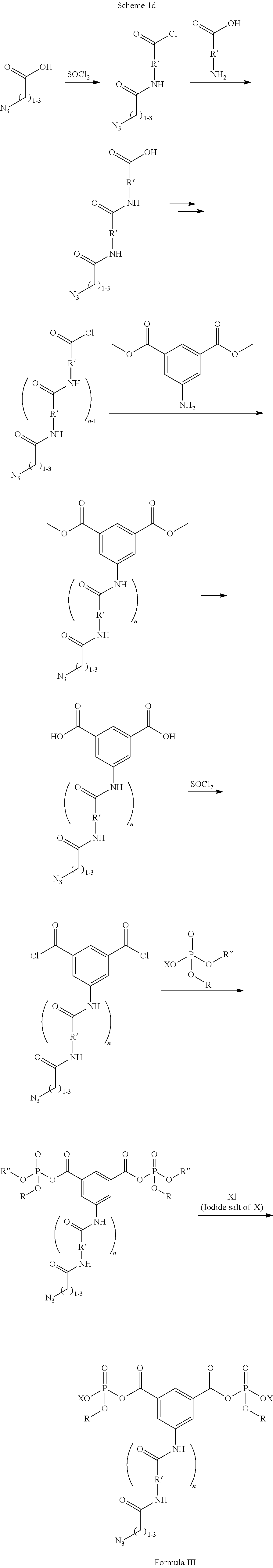

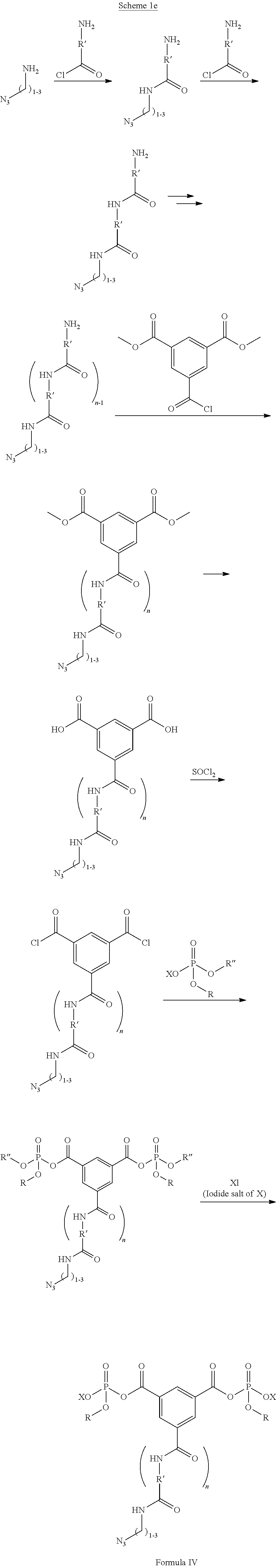

In each of Schemes 1a to 1e, R″ is the same as or different from R and X is an alkali ion; and the double arrows represent repeating steps 1 and 2 to extend the azide-containing moiety to a specified hydrocarbon chain length where n=1 to 5.

In yet another aspect, there is provided a cross-linked hemoglobin protein comprising a hemoglobin protein cross-linked by an azide-containing cross-linker disclosed herein, wherein the hemoglobin protein comprises two alpha and two beta subunits. In one example, each of the beta subunits of the hemoglobin protein is covalently linked via an amide bond to the azide-containing cross-linker. In another example, the cross-linked hemoglobin protein is represented by Formula VI

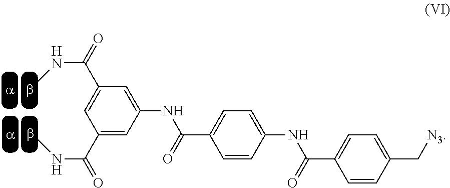 [SMILES: CNC(=O)c1cc(NC(=O)c2ccc(NC(=O)c3ccc(CN=[N+]=[N-])cc3)cc2)cc(C(=O)NC)c1]

In yet another aspect, there is provided a hemoglobin-based oxygen carrier (HBOC), comprising a pair of the cross-linked hemoglobin disclosed herein coupled by a copper catalyzed azide-alkyne cycloaddition (CuAAC). In one embodiment, the HBOC is represented by Formula VII

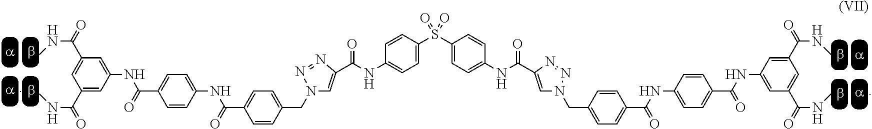

In yet another aspect, there is provided a composition for use to increase oxygen transport, comprising a HBOC disclosed herein and a suitable carrier. In one embodiment, such composition can be used in perfusion or transfusion.

Other features and advantages of the present disclosure will become apparent from the following detailed description. It should be understood, however, that the detailed description and the specific examples while indicating preferred embodiments are given by way of illustration only, since various changes and modifications within the spirit and scope of the present disclosure will become apparent to those skilled in the art from this detailed description.

### BRIEF DESCRIPTION OF THE DRAWINGS

FIG. 1 is a ¹H Nuclear magnetic resonance (NMR) spectrum of an azide-containing cross-linker in accordance with an embodiment of the present disclosure.

FIG. 2 is a ³¹P NMR spectrum of the azide-containing cross-linker of FIG. 1.

FIG. 3 is a mass spectrum of a cross-linked carbonmonoxy hemoglobin-azide in accordance with another embodiment of the present disclosure.

FIG. 4 shows a reverse-phase HPLC trace of the cross-linked carbonmonoxy hemoglobin-azide of FIG. 3 under dissociating conditions before heat treatment.

FIG. 5 shows a reverse-phase HPLC trace of the cross-linked carbonmonoxy hemoglobin-azide of FIG. 3 under dissociating conditions after heat treatment.

FIG. 6 shows a size-exclusion fast protein liquid chromatography (FPLC) trace of a HBOC after CuAAC reaction in accordance with yet another embodiment of the present disclosure.

FIG. 7 shows oxygen binding curves of the HBOC (shown as “bis-tetramers”) of FIG. 6.

FIG. 8 shows a reverse-phase HPLC trace of a cross-linked carbonmonoxy hemoglobin-azide prepared in accordance with yet another embodiment of the present disclosure under dissociating conditions before heat treatment.

FIG. 9 shows a reverse-phase HPLC trace of the cross-linked carbonmonoxy hemoglobin-azide of FIG. 8 under dissociating conditions after heat treatment.

### DETAILED DESCRIPTION

As used herein, the expression “capable of binding oxygen” is meant to refer to the property of the HBOCs disclosed in the present disclosure that such HBOCs would have oxygen carrier capabilities. It is expected that the HBOCs described herein are capable of binding oxygen under an atmosphere that is at a high partial pressure of oxygen and releasing it at a lower partial pressure of oxygen in the circulation.

The term “hemoglobin” as used herein not only relates without limitation to human adult Hemoglobin A, but also includes any hemoglobin from any source.

The term “moiety” as used herein is meant to refer to a part of an organic molecule.

The term “functional group” as used herein generally refers to an atom, or a group of atoms that has similar chemical properties whenever it occurs in different compounds. Functional groups define the characteristic physical and chemical properties of families of organic compounds. The functional groups referred to in the present disclosure have the meaning that is generally known in the art.

The term “isomer”, including its adjectives, as used herein generally refers to compounds or molecules having the same molecular formula but different arrangements of atoms within the molecules.

The term “derivative” as used herein generally refers to a compound or molecule that is derived from a parent compound or molecule by a chemical or physical process. As can be appreciated by those skilled in the art, a derivative generally includes multiple isomers.

The chemical, biological and pharmaceutical terms used herein have meanings generally known in the art. Those skilled in the art are also familiar with the meaning of these terms.

### Azide-Containing Cross-linkers and Methods of Preparation

According to one aspect, the present disclosure provides novel azide-containing cross-linkers that are capable of forming covalent bonds with the β subunits of a hemoglobin tetramer, resulting in a β-β cross-linked hemoglobin-azide.

In one embodiment, the azide-containing cross-linker is a compound of Formula I, II, III or IV shown below:

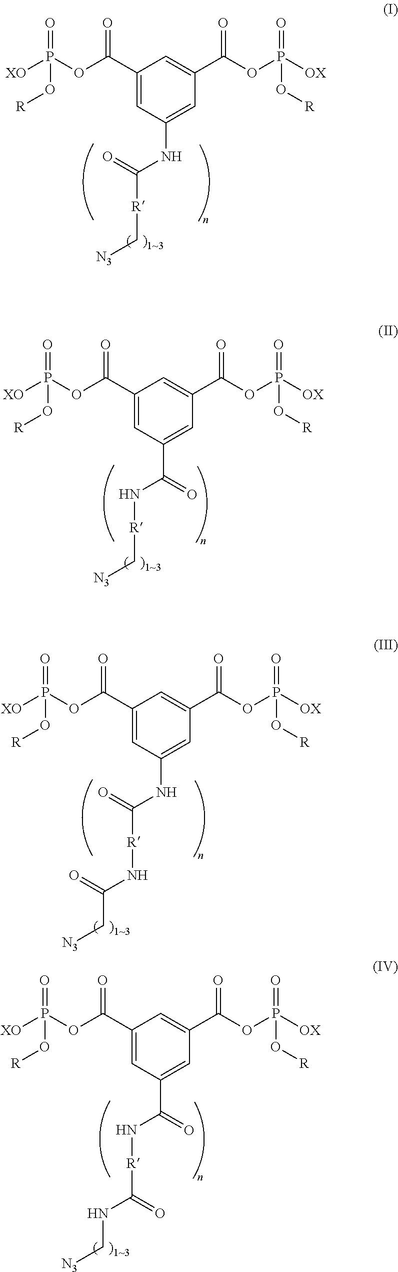

In each of Formula I to IV, R is an alkyl; X is an alkali ion; and n is 1 to 5. In some embodiments, R is for example methyl or ethyl. In some embodiments, X is a lithium or sodium ion. In some embodiments, n is 2 to 5.

In some embodiments, in each of Formula I to IV, R′ is a benzene or benzene

isomer and such benzene or benzene isomer can be either para- or meta-substituted. Using R′=benzene as an example, depending on the position of the amide bond connecting to R′ in Formula I to IV, the para- or meta-substituted R′ is respectively

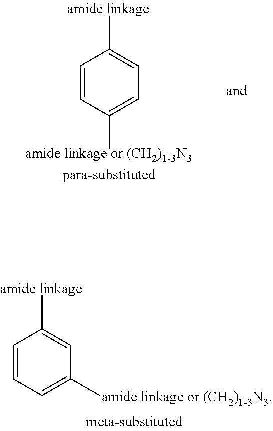

In some embodiments, in each of Formula I to IV, R′ is a heterocyclic six-membered aromatic ring or its isomers, which are optionally either para- or meta-substituted. Exemplary heterocyclic six-membered aromatic rings include the following:

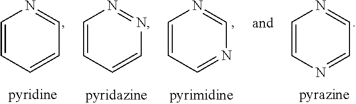

In some embodiments, in each of Formula I to IV, R′ is a five-membered aromatic ring or a derivative thereof, including isomers for each derivative, all of which are optionally substituted, for example, 1,3 substituted. Exemplary five-membered aromatic rings include the following:

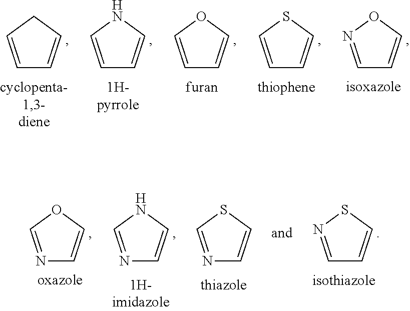

In some embodiments, in each of Formula I to IV, R′ is an indole ring or a derivative thereof, including isomers for each derivative. Exemplary indole ring and its derivatives include the following:

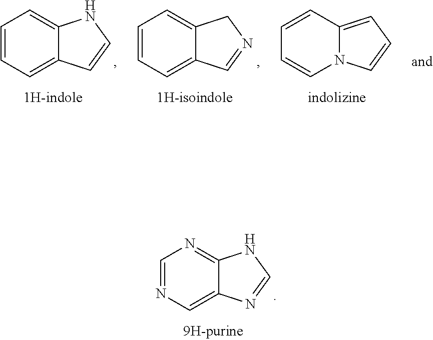

In some embodiments, in each of Formula I to IV, R′ is naphthalene or a derivative thereof, including isomers for each derivative. Exemplary naphthalene and its derivatives include the following:

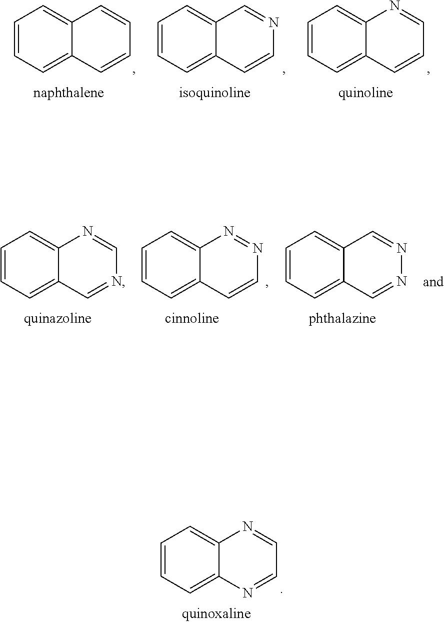

In some embodiments, R's having at least one aromatic ring are preferred. It is postulated that such R's can provide a proper steric conformation or rigidity to allow the azide cross-linker to stay rigid for its reactivity in a CuAAC process.

In some embodiments, at least one of the aromatic rings in R′ is mono-, di-, tri- or tetra substituted with a methyl, ethyl, allyl, propyl, isopropyl, fluoro, chloro, bromo, iodo, hydroxyl, methoxy, ethyoxy, isopropyl, carboxyl, acetyl, cyano, or nitro group.

In some embodiments, the azide moiety at the end of the cross-linker of any one of Formula I to IV is azidomethyl.

In some embodiments, at least one aromatic ring of R′ of the cross-linker of any one of Formula I to IV is mono-, bis- or tris substituted with an azide moiety

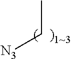

at the end of the cross-linker, for example:

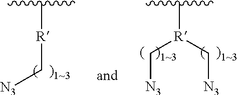

(if R′ allows tri-substitution). Hence, in some instances, the azide cross-linker is:

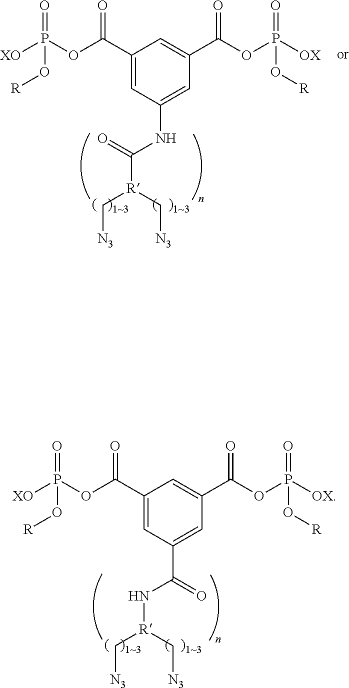

For example, when R′ is benzene and n=1, the azide cross-linker is:

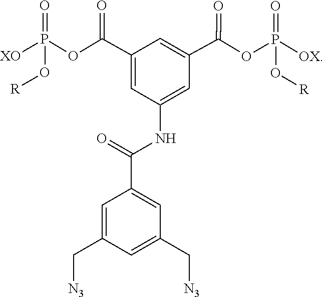

In other instances,

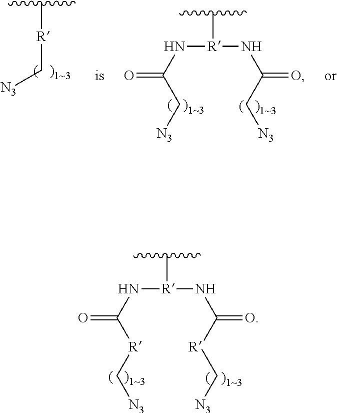

In one embodiment, the novel azide-containing cross-linker is a compound of Formula V

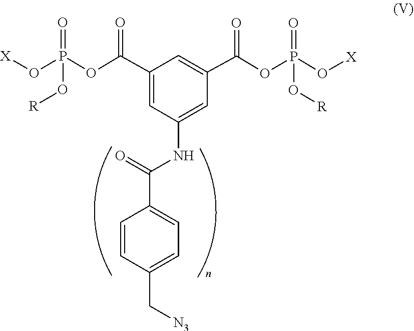

wherein R is an alkyl, for example, methyl or ethyl;
X is an alkali ion, for example lithium or sodium ion; and
n is 1 to 5. In some embodiments, n is 2 to 5.

An exemplary cross-linker can have the following structure:

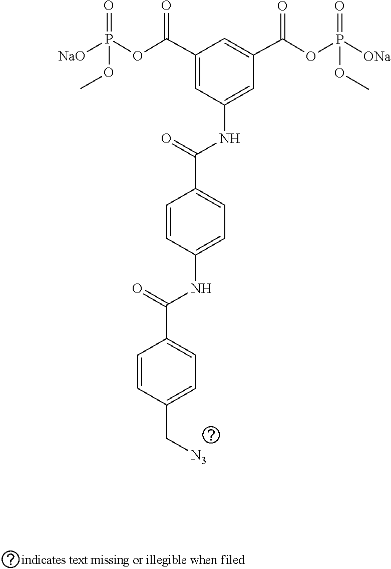

While not wishing to be bound by any theory, it is believed that extending the location of the azide group farther from the hemoglobin protein when the azide cross-linker is covalently linked to the protein would make the cross-linked protein more readily available to combine with an alkyne, for example, a bis-, tris- or terakis-alkyne in a CuAAC process to form a bis-tetramer HBOC. In one embodiment, a novel azide-containing cross-linker of the present disclosure contains an azide moiety that is farther from the cross-linking sites of the cross-linker (that is farther from the protein's chains). This has allowed the azide moiety of the cross-linked hemoglobin-azide to become more accessible to react with the alkyne moiety of the alkyne in a CuAAC process to produce the bis-tretramer in high yields.

It is hypothesized that protein-protein CuAAC proceeds only where the azide-containing cross-linker is installed between readily accessible lysyl ε-amino groups within the 2,3-DPG (2,3-diphosphoglycerate) binding site of the β subunits of the hemoglobin protein, specifically at the two βLys82 ε-amino groups. The 2,3-DPG binding site, being highly cationic, can be targeted by anionic cross-linkers; however, competing reactions may occur between the two αLys99 ¿-amino groups. As the αα-azide-containing cross-linked hemoglobin does not undergo CuAAC, it also prevents formation of the βlys82 cross-linked hemoglobin-azide. In order to utilize HBOC bis-tetramers on a larger scale for use in a variety of applications, it would be desirable to have a more efficient route: particularly, one that avoids cross-linking between the a subunits.

Acyl phosphates are anionic acetylating agents and highly polar with a high anionic charge density. They are also water soluble and similar to biological molecules. While not wishing to be bound by any theory, it is believed that by incorporating an acyl phosphate into the azide-containing cross-linker, the high reactivity of the acyl phosphate moiety would make the cross-linker more β-selective, thus improving the overall yield of the HBOC bis-tetramer. As the hemoglobin protein contains two β subunits, it is necessary that the azide-containing cross-linker comprises at least two terminal phosphate moieties to allow reactions to be site-selective for reactions at the cationic sites of the two β subunits.

In one embodiment of the present disclosure, the azide-containing cross-linker is functionalized by bis acyl phosphate that forms amide bonds with the two β subunits, resulting in the formation of a cross-linked hemoglobin-azide.

Examples of suitable acyl phosphates include acyl alkyl phosphates. In one embodiment, the acyl phosphate is an acyl methyl phosphate, and in another embodiment, the alkyl phosphate is an acyl ethyl phosphate.

The azide-containing cross-linkers of the present disclosure can be prepared in accordance with one of the reaction Schemes 1a to 1e described herein or an analogous scheme thereof. The precursors to the cross-linkers can be prepared using commercially available materials and/or following synthetic methods known in the art.

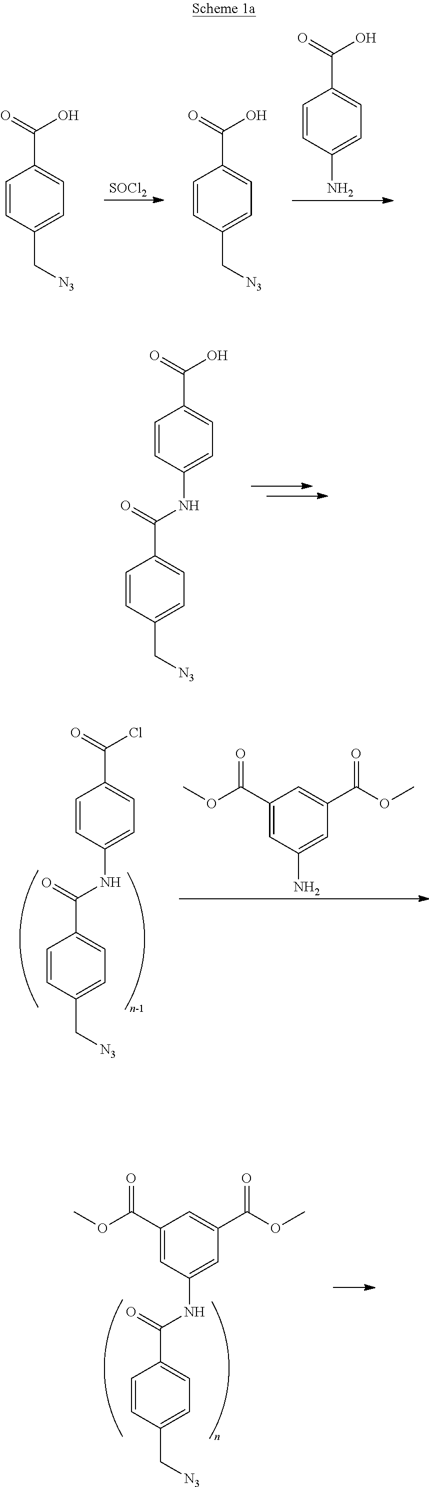

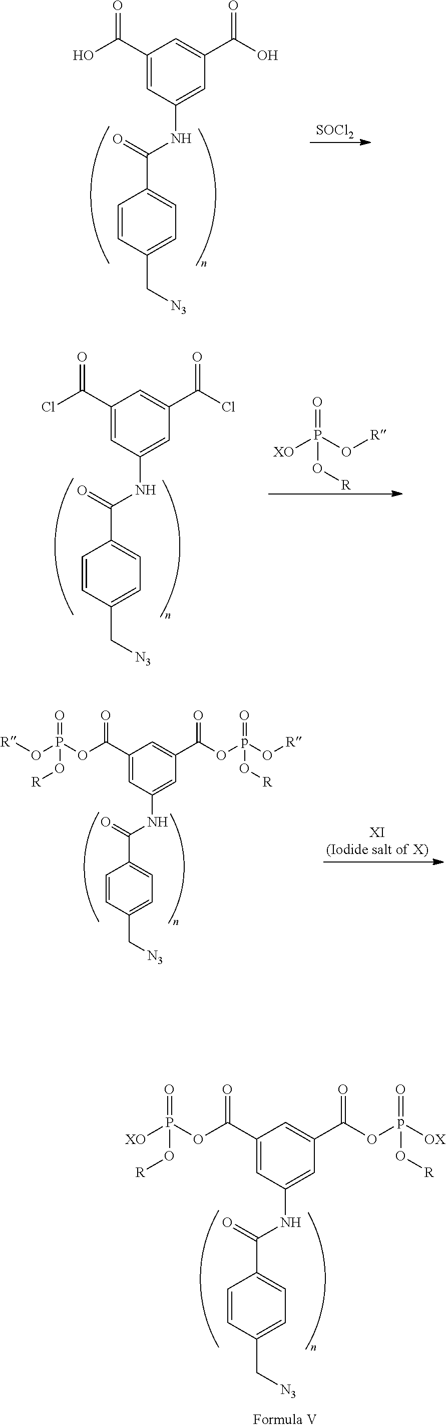

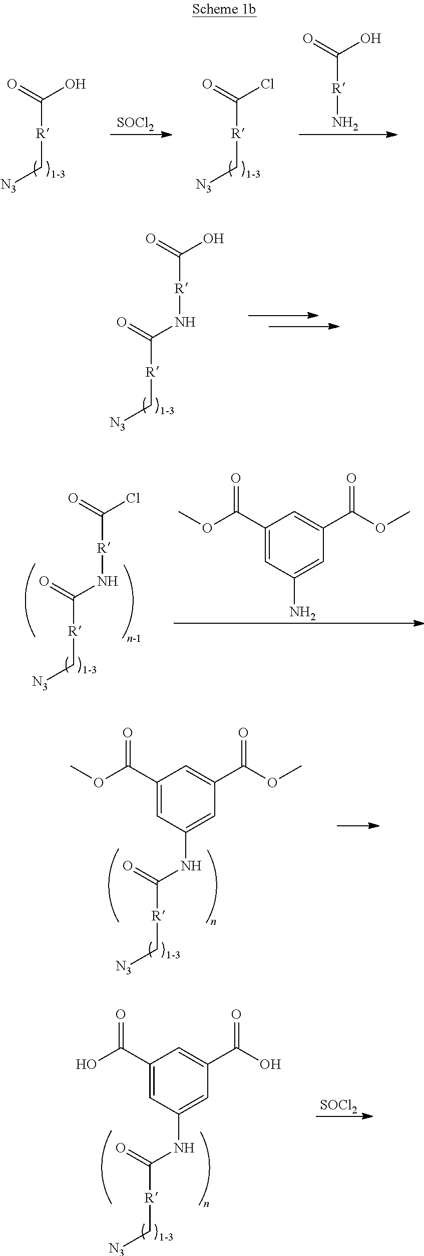

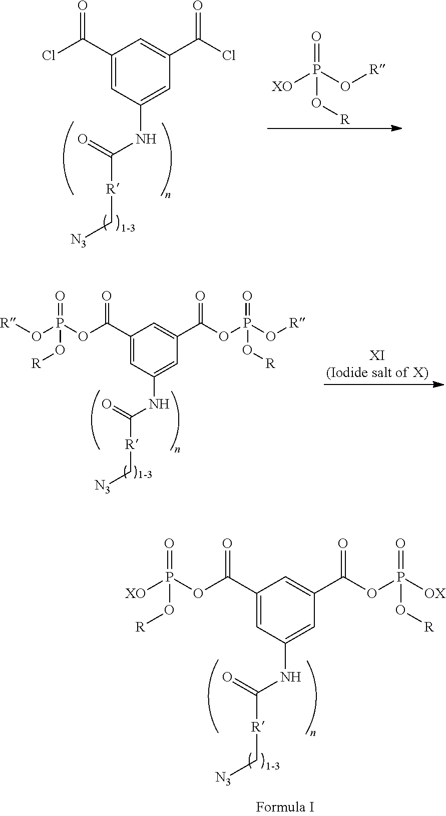

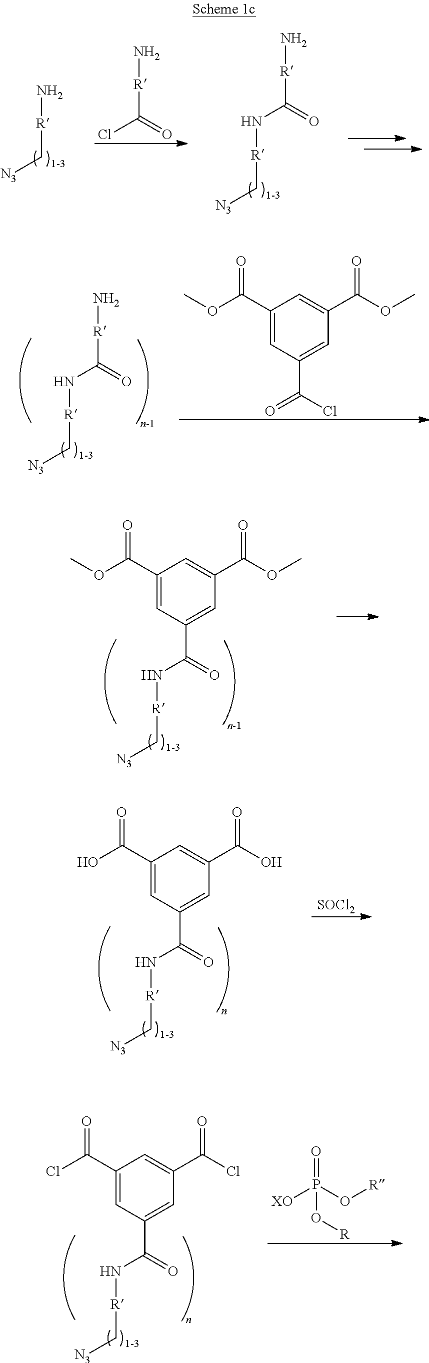

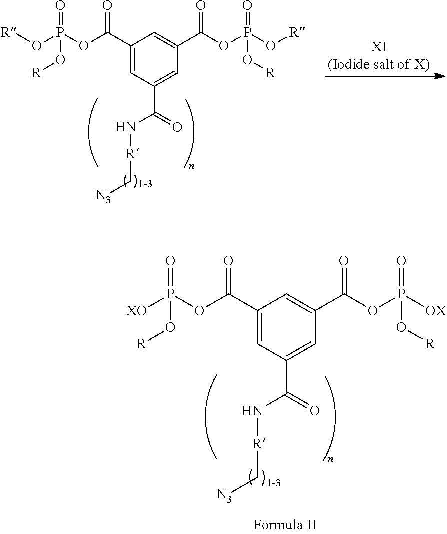

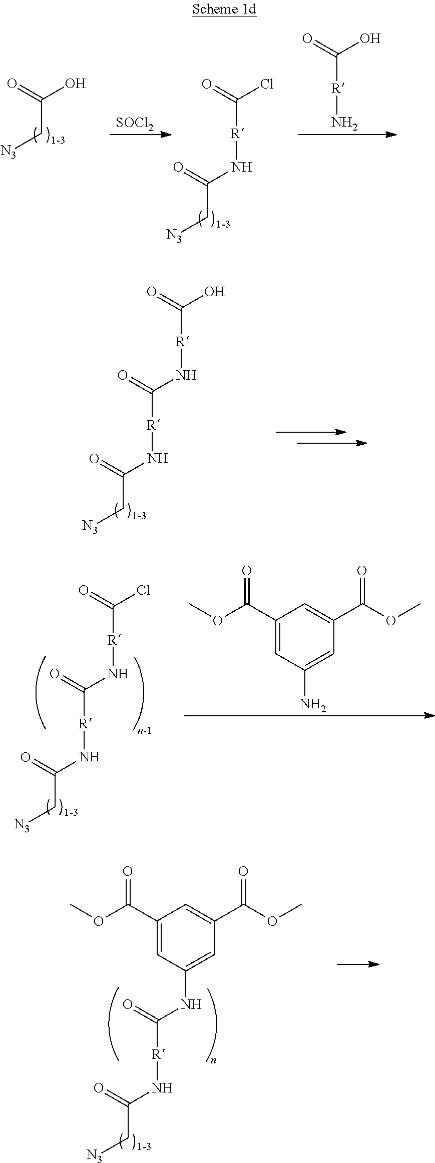

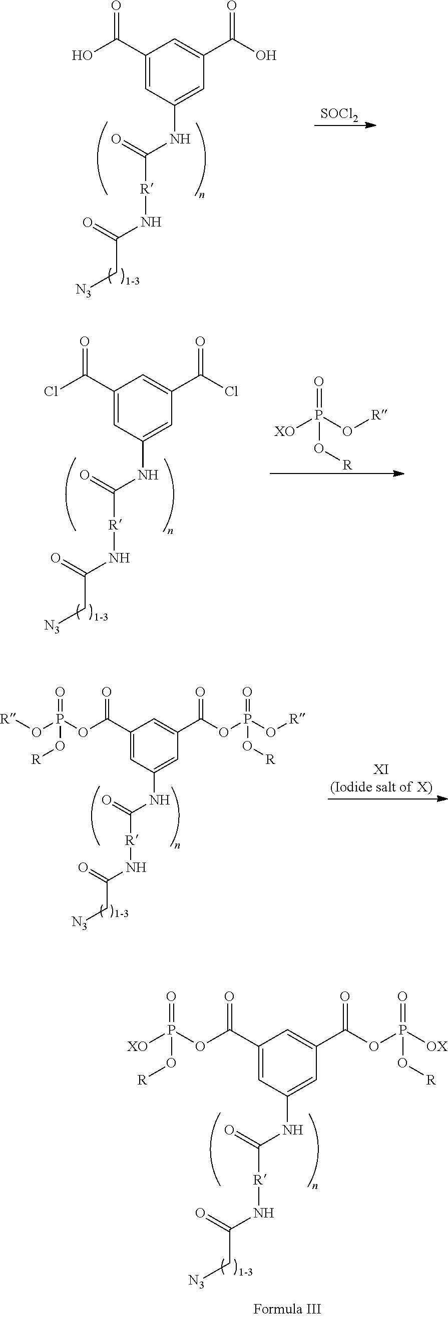

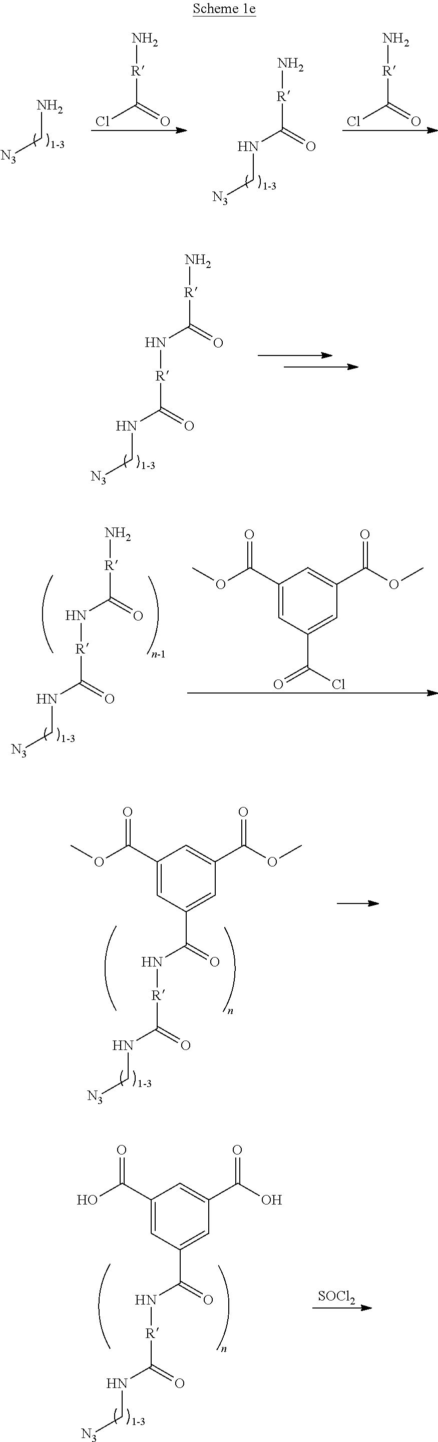

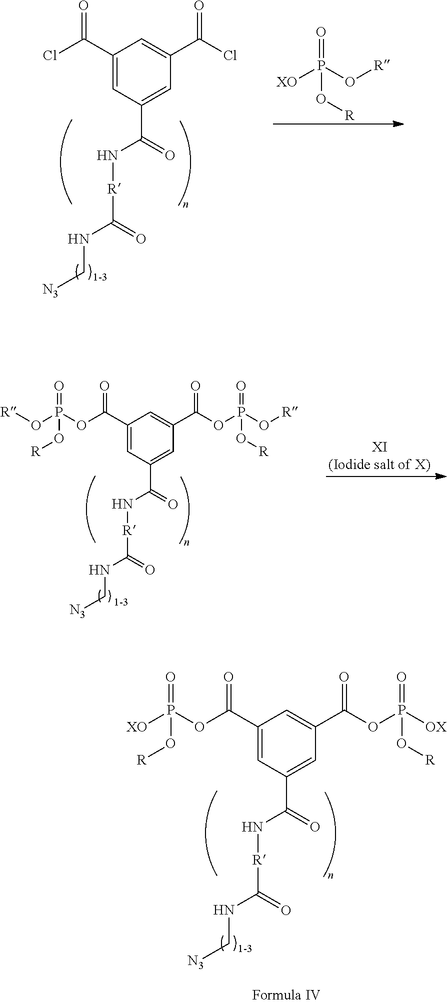

In each of Schemes 1a to 1e, R″ is the same as or different from R, and the first two steps can be repeated to extend the azide-containing moiety to a specified hydrocarbon chain length moiety, wherein n is 1 to 5. Although XI, iodide salt of X, is depicted in the last reaction step in these schemes, another suitable halide of X may be used. In one embodiment, an alkali metal iodide, for example, NaI, is preferred. In another embodiment, an alkali metal bromide may also be used. In some embodiments, R″ is an alkyl such as methyl or ethyl.

In one exemplary process, 1-benzamido-4-methyl-azide is added to 4-aminobenzoic acid to form 4-(4-(azidomethyl)benzamido) benzoic acid (shown below):

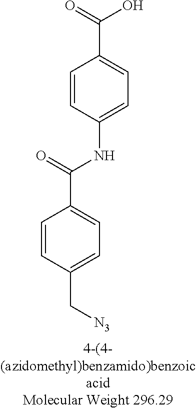 [SMILES: [N-]=[N+]=NCc1ccc(C(=O)Nc2ccc(C(=O)O)cc2)cc1]

The 4-(4-(azidomethyl)benzamido) benzoic acid is then converted to dimethyl 5-(4-(4-(azidomethyl)benzamido) benzamindo) isophthalic acid so that acyl methyl phosphate can be incorporated into the cross-linker, as shown in the reaction scheme below:

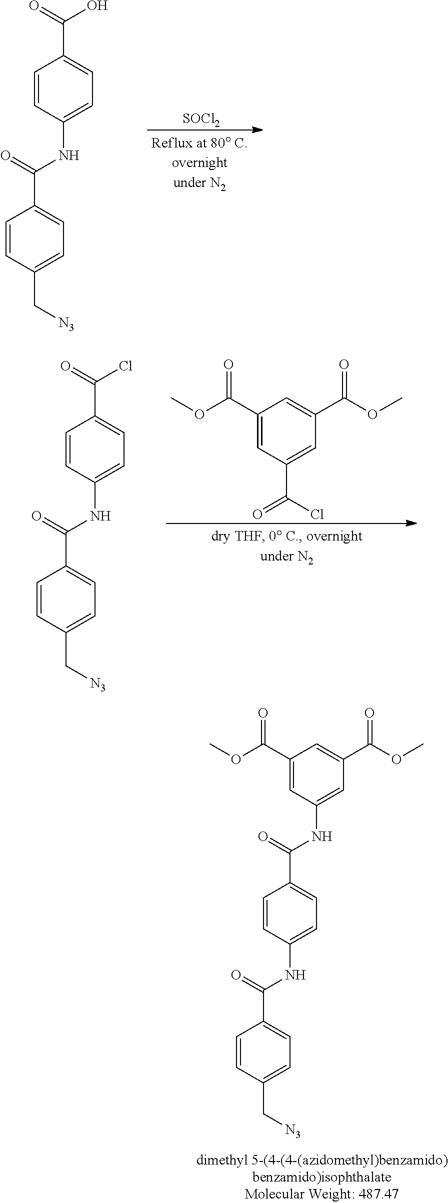

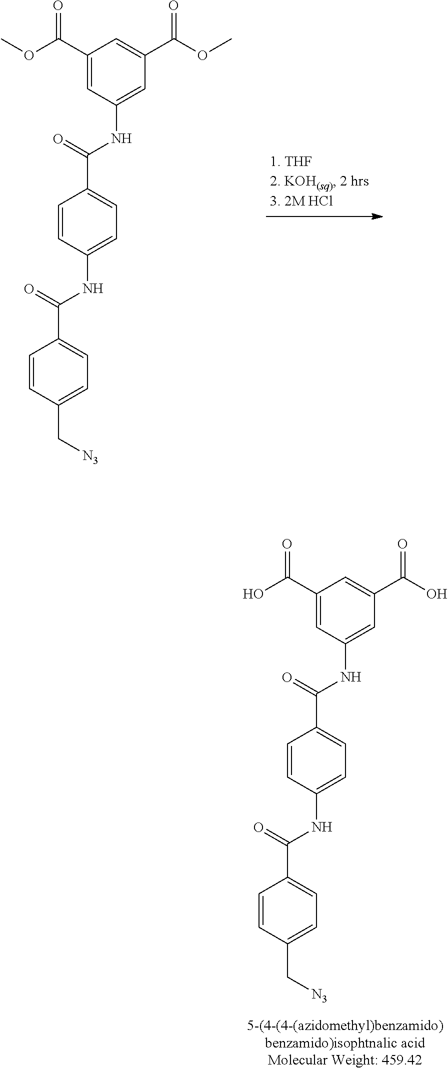

Lastly, a bis(acyl methyl phosphate) azide cross-linker (in this embodiment, bis(sodium methyl phosphate)) is prepared in accordance with the reaction scheme below:

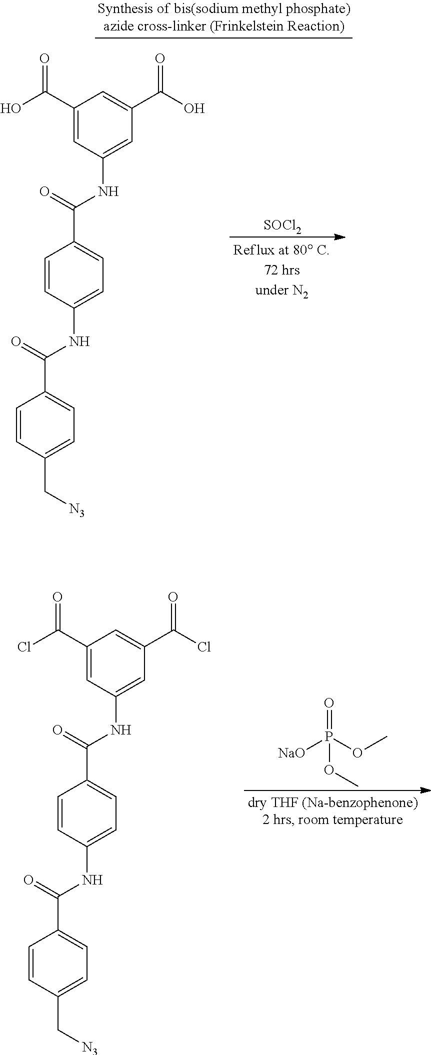

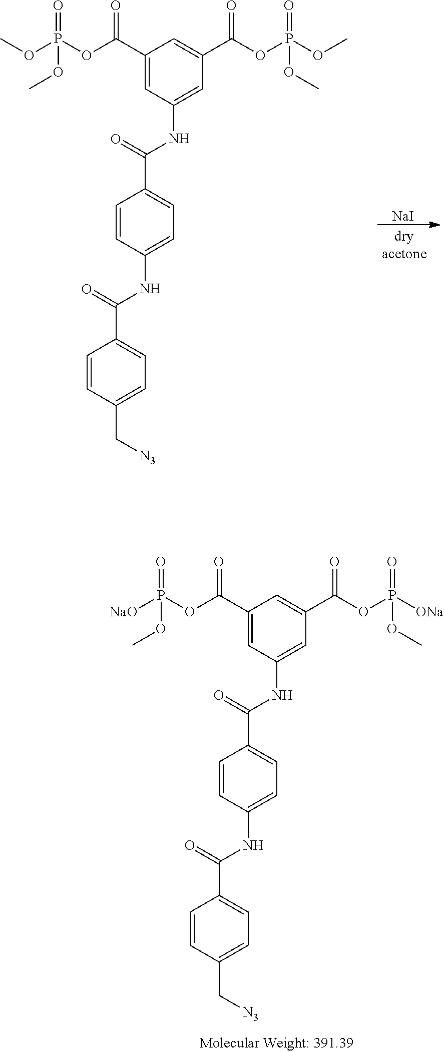

The bis(acyl methyl phosphate) azide cross-linker thus obtained can selectively bind to the β subunits of hemoglobin, resulting in the formation of the βlys82 cross-linked hemoglobin-azide.

The term “alkyl” as used herein, whether it is used alone or as part of another group, means straight or branched chain alkyl groups.

The term “acyl” as used herein, whether it is used alone or as part of another group, means straight or branched chain acyl groups.

### Cross-Linked Hemoglobin-Azide

According to one aspect, the present disclosure provides a novel cross-linked hemoglobin-azide, wherein the novel azide-containing cross-linker selectively binds the β subunits of the hemoglobin by forming an amide with each of the β subunits.

In one embodiment, the novel azide-containing cross-linker of the present disclosure is incorporated into the hemoglobin tetramer having the azide moiety away from the protein. An exemplary cross-linked hemoglobin-azide is shown below as Formula VI:

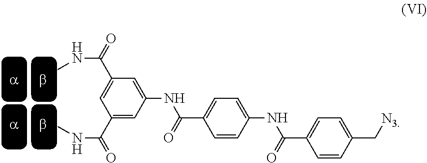 [SMILES: CNC(=O)c1cc(NC(=O)c2ccc(NC(=O)c3ccc(CN=[N+]=[N-])cc3)cc2)cc(C(=O)NC)c1]

In some embodiments, the hemoglobin is deoxy hemoglobin, carbonmonoxy hemoglobin or oxy hemoglobin. An exemplary hemoglobin is carbonmonoxy hemoglobin.

The cross-linked hemoglobin-azide of the present disclosure may be prepared by modifying methods described in, including but not limited to Wang, A.; Kluger, R., Increasing efficiency in protein-protein coupling: subunit-directed acetylation and phase-directed CuAAC (“click coupling”) in the formation of hemoglobin bis-tetramers. Biochemistry 2014, 53 (43) 6793-(“Wang”) and the references cited therein, which are incorporated herein by reference in their entirety. In an exemplary process, the crude cross-linked hemoglobin-azide may undergo heat treatment to remove uncross-linked hemoglobin moieties.

Cross-linking yields from using the azide-containing cross-linker of the present disclosure can be assessed by known methods, including size-exclusion HPLC using a high-salt concentration elution buffer (for example, 0.5 M MgCl₂). Under these conditions, proteins lacking cross-linkers dissociate into 32 kDa αβ-dimers or elute after the cross-linked β-β tetramer (eluting at 64 kDA). In some embodiments, the cross-linking yields can range between 40-50%.

The cross-linked hemoglobin-azide of the present disclosure can be readily

available to combine with an alkyne in a CuAAC process to form a bis-tetramer HBOC.

### Bis-Tetramer HBOC by CuAAC

According to one aspect, the present disclosure provides a novel HBOC comprising a pair of the cross-linked hemoglobin-azide coupled by a CuAAC process. The HBOC of the present disclosure is a stable entity that binds and releases oxygen and is designed to minimize any side effects related to blood pressure associated with depletion of endogenous nitric oxide.

An exemplary bis-tetramer HBOC is shown below in Formula VII:

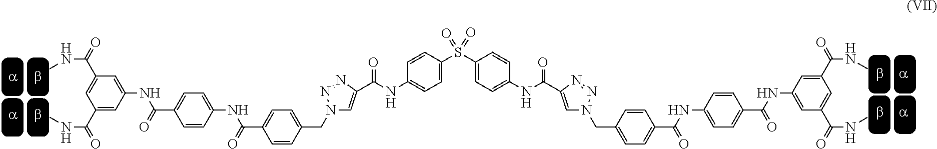

To assess the ability of the bis-tetramer HBOC to transport oxygen, its oxygen binding properties can be measured by analytical methods known in the art. For example, oxygen dissociation curves can be generated by fitting the data obtained from an analyzer, such as the Hemox Analyzer™, to the Adair equation. This gives the oxygen affinity as P₅₀, pressure of oxygen needed for saturation of half the heme sites. The cooperativity (n₅₀) can also be calculated, which indicates the likelihood of the bis-tetramer functioning as an effective oxygen carrier in circulation.

Exemplary bis-tetramers of the present disclosure exhibit significant oxygen affinity (n₅₀=1.5±0.2; P₅₀=4.0±0.8 torr), when compared to that of native hemoglobin (n₅₀=2.7±0.2; P₅₀=9.1±0.6 torr). The significant cooperativity suggests that the bis-tetramers will likely be able to function as an effective oxygen carrier in circulation.

The bis-tetramer HBOC of the present disclosure can be prepared by analougs methods known in the art, particularly by coupling a pair of the cross-linked hemoglobin-azides of the present disclosure via an alkyne using a CuAAC process.

It has been proposed that CuAAC forms conjugates of proteins through a selective and rapid combination of two bioorthogonal functional groups, one of which must be introduced into the protein. Therefore, CuAAC can be utilized in the formation of structurally defined coupled proteins. Such a process can be efficient where it utilizes serial or sequential addition of protein-derived azides to a compound with two or more terminal alkynes. Therefore, it is believed that the CuAAC can be employed in the formation of the bis-tetramers of the present disclosure. Methods analogous to those disclosed in Wang may be applied to prepare the bis-tetramers of the present disclosure. In an exemplary process, a suitable alkyne has dual terminal alkyne moieties to allow a sequential coupling of the cross-linked hemoglobin-azides. Examples of the alkyne include a bis-alkyne such as

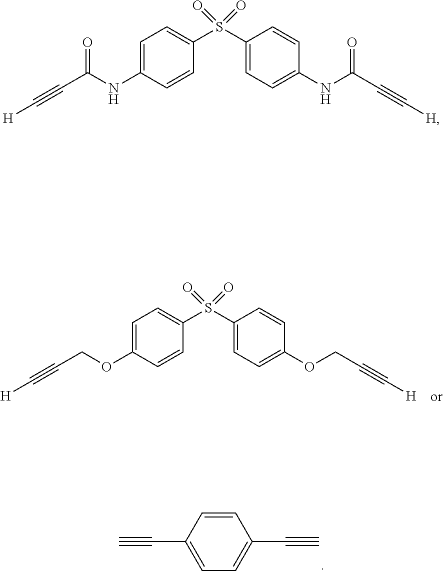

In one embodiment, the bis-alkyne is

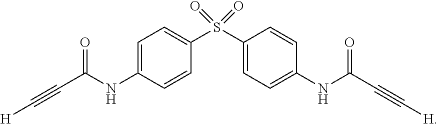

In some embodiments, tris-alkynes or tetrakis-alkynes can be used as they provide an additional site for reaction in the event of hydrolysis of one of the acyl phosphates. An exemplary tris-alkyne is

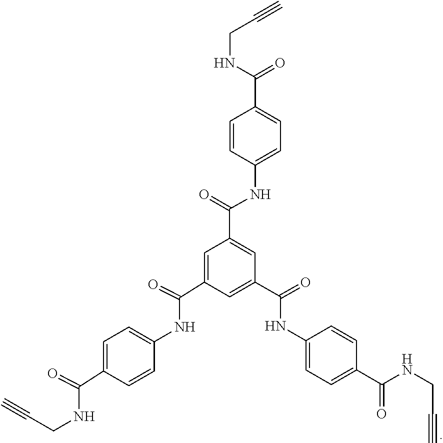 [SMILES: C#CCNC(=O)c1ccc(NC(=O)c2cc(C(=O)Nc3ccc(C(=O)NCC#C)cc3)cc(C(=O)Nc3ccc(C(=O)NCC#C)cc3)c2)cc1]

An exemplary tetrakis-alkyne is

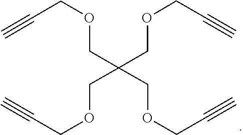 [SMILES: C#CCOCC(COCC#C)(COCC#C)COCC#C]

It will be appreciated that a suitable alkyne for the CuAAC process will have at least two terminal alkyne moieties. Preferably, the at least two terminal alkyne moieties are far away enough to avoid steric hindrance.

In an exemplary process, a phase-directed CuAAC is employed to prepare the bis-tetramer. In this process, it is assumed that a first cross-linked hemoglobin-azide of the present disclosure is conjugated or coupled with a first alkyne moiety of the alkyne, which is sufficiently insoluble, and the resulting conjugate becomes soluble and conjugates or couples with a second cross-linked hemoglobin-azide at a faster rate, ensuring that the second cross-linked hemoglobin-azide reacts. Thus, the two sequential steps of the coupling process can be cooperative and would permit the use of greater than stoichiometric amounts of the alkyne linker with assured coupling to form the bis-tetramer. In some instances, the molar equivalent of the alkyne to the cross-linked hemoglobine-azide is about 3 to 10.

The yields of bis-tetramers can be measured by size-exclusion HPLC. It is expected that the increased B subunit selectivity due to the application of the azide-containing cross-linkers disclosed herein would effectively translate into a high yield of the bis-tetramer. In some embodiments, the overall yield of the bis-tetramer using the azide-containing cross-linkers is about 70-72%. In other embodiments, the overall yield can be greater than 80%.

The bis-tetramer can be further purified via column chromatography to remove

copper from the CuAAC process. For example, the crude production solution may be purified using a G100 Superdex™ column to remove residual copper should that be required.

Table 1 shows yields of exemplary HBOCs prepared in accordance with Scheme 1a.

TABLE 1

| | Cross-linking Yield | | |
|---|---|---|---|
| n in Formula (I) | (β-β Cross-linker) | CuAAC Yield | Overall Yield |
| 1 | 40% | 65% | 26% |
| 2 | 40% | 72% | 29% |

As shown in Table 1, the exemplary CuAAC processes show a 65 to 72% conversion to HBOC bis-tetramers from the β-β azide cross-linker. When n=2, the CuAAC yield has increased from 65% to 72%, which represents an almost 11% increase when compared with n=1. It is postulated that this outcome can be due to the β-selective cross-linking followed by a heat treatment to remove impurities including non-cross-linked proteins, such as native hemoglobin or singly modified hemoglobin, thereby avoiding contamination prior to the CuAAC process and significantly increasing the yield for the CuAAC process (an almost 12% increase when comparing n=2 with n=1). In addition, the processes described herein can be scaled up using large mixing tanks.

In one embodiment, the bis-tetramer HBOC of the present disclosure may be incorporated into a composition together with a suitable carrier. The composition may be used as an oxygen carrier in transplant perfusion or transfusion. The term “carrier” as used herein refers to, for example, a diluent, adjuvant, excipient, auxiliary agent or vehicle which may be combined with an active agent of the present disclosure.

Embodiments of the present disclosure will now be described by way of examples.

All reagents used in the Examples below were obtained from commercial suppliers and were used without further purification unless otherwise indicated. Highly purified human hemoglobin had been produced by Hemosol Inc. and was supplied by successor companies.

### Example 1

### Synthesis of Azide-Containing Bis-(Acyl Phosphate Monoester) Cross-Linker (8).

Azide-containing bis-(acyl phosphate monoester) cross-linker was synthesized in accordance with Scheme 2 below.

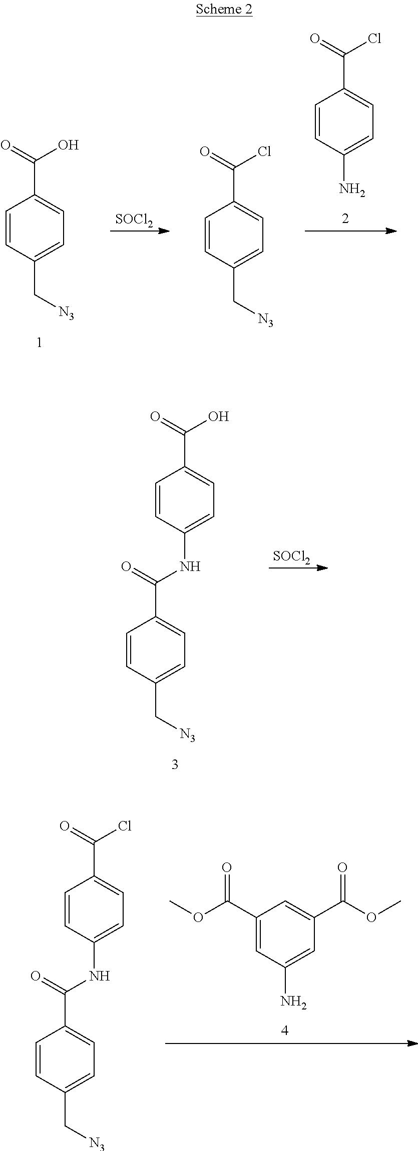

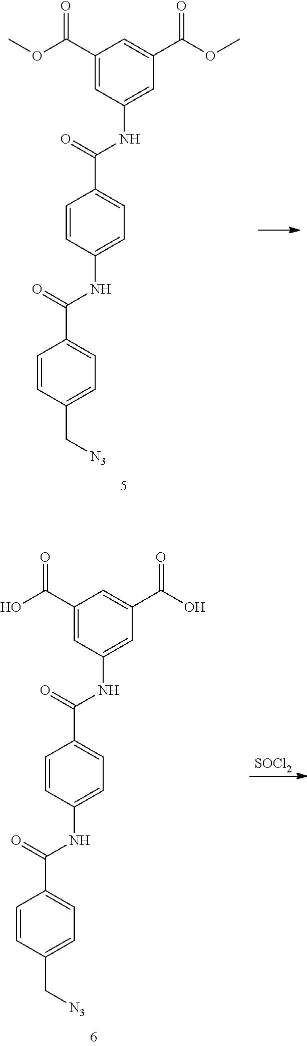

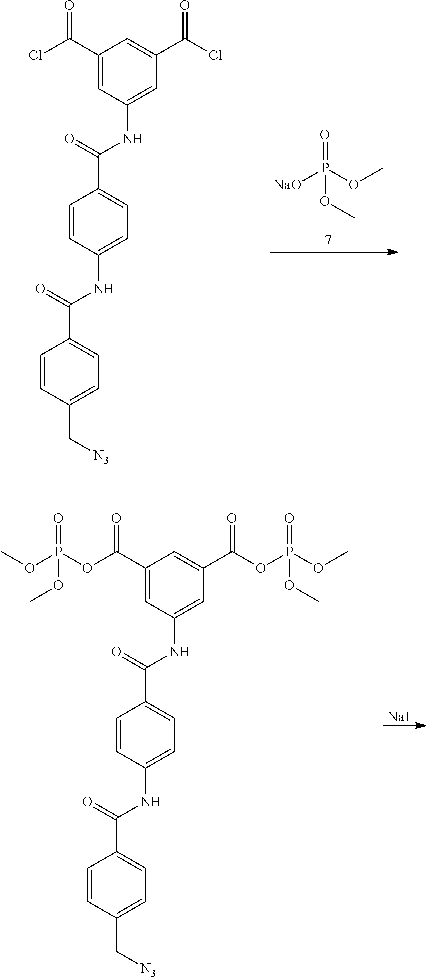

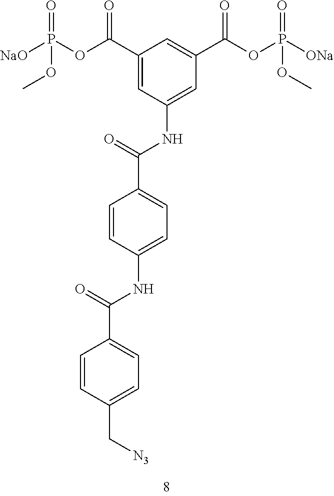

4-(azidomethyl)benzoic acid (1; 0.48 g; 2.7 mmol) was refluxed in excess thionyl chloride (SOCl₂) overnight under nitrogen gas to yield the corresponding acyl chloride. Residual thionyl chloride was evaporated thereby leaving a yellow oil.

The yellow oil was dissolved in dry tetrahydrofuran (THF) and the THF solution was added dropwise to a stirred solution of 4-aminobenzoic acid (2; 0.39 g; 2.8 mmol) with dry THF at 0° C. under nitrogen. The reaction solution was stirred for four hours as the flask containing the solution warmed to room temperature. A solution of dilute hydrochloric acid was added, and the product was extracted with ethyl acetate. The organic layer was washed with brine and dried over anhydrous magnesium sulfate, which was removed by vacuum filtration. The solvent was then evaporated, leaving a pale-yellow solid, 4-(4-(azidomethyl)benzamido) benzoic acid (3).

The crude 4-(4-(azidomethyl)benzamido) benzoic acid (3) was purified by column chromatography using a 10% methanol in dichloromethane with 0.1% trifluoroacetic acid as the solvent system.

The purified 4-(4-(azidomethyl)benzamido) benzoic acid (3) (0.10 g; 0.38 mmol) was added to excess (about 15 mL) thionyl chloride and refluxed under nitrogen to form a clear yellow solution. After 24 hours, the reaction mixture was cooled to room temperature and residual thionyl chloride was removed by rotary evaporation, leaving a pale-yellow solid, which was an acyl chloride. The acyl chloride product was immediately dissolved in dry THF. The reaction mixture was added dropwise to a stirred solution of dimethyl 5-aminoisophthalate (4; 79 mg; 0.38 mmol) in dry THF under nitrogen at 0° C. The reaction was stirred for 4 hours as it warms to room temperature. Double distilled (dd) H₂O (60 mL) was added and the pale-yellow precipitate was extracted with ethyl acetate (3×40 mL). The organic layer was washed with brine and dried over anhydrous magnesium sulfate. Magnesium sulfate was is removed by vacuum filtration and the solvent was evaporated to obtain a yellow solid, dimethyl 5-(4-(4-(azidomethyl)benzamido) benzamindo) isophthalate (5), which was purified by column chromatography using a 1:1 ethyl acetate:hexane solvent system. 5 was suspended in a 1:1 THF:methanol mixture.

A solution of potassium hydroxide (5 g in 15 mL of ddH₂O) was prepared separately and added dropwise to the suspension containing 5 over 10 minutes to yield a clear yellow solution. After 2 hours, 2 M hydrochloric acid was added slowly until the pH reached about 2. A white precipitate was extracted with ethyl acetate (3×40 mL) and the organic layer was washed with brine and dried over anhydrous magnesium sulfate. The mixture was filtered, and the solvent was removed by rotary evaporation to obtain 5-(4-(4-(azidomethyl)benzamido) benzamindo) isophthalic acid (6).

6 (54 mg; 0.12 mmol) was added to excess thionyl chloride (5 mL) and refluxed under nitrogen for 72 hours to obtain a clear yellow solution. Once the mixture was cooled to room temperature, residual thionyl chloride was removed by rotary evaporation to obtain a pale-yellow solid. Then, sodium dimethyl phosphate (7; 87 mg; 0.59 mmol) was added to a flask containing the diacyl chloride product and dry THF (10 ml) prepared from sodium-benzophenone distillation was slowly dropped into the flask while stirred in sealed flask under nitrogen. After 3 hours, the mixture was filtered by vacuum to remove excess sodium dimethyl phosphate 7.

The resulting filtrate was dropped directly into a round-bottom flask containing sodium iodide (71 mg; 0.47 mmol) dissolved in dry acetone. The azide-containing bis-(acyl phosphate monoester) cross-linker (8) was allowed to precipitate overnight and collected by vacuum filtration. Cold and dry acetone was used to wash the pale-yellow solid, which was analyzed by NMR spectroscopy and mass spectroscopy without further purification.

Yield=95%.

¹H NMR (400 MHZ, DMSO) δ 10.61 (s, 1H), 10.58 (s, 1H), 8.71 (d, J=2.9, 1.6 Hz, 2H), 8.22 (t, J=1.6 Hz, 1H), 8.06 (d, J=8.9 Hz, 2H), 8.03 (d, J=8.4 Hz, 2H), 7.97 (d, J=8.8 Hz, 2H), 7.55 (d, J=8.2 Hz, 2H), 4.58 (s, 2H), 3.53 (d, J=11.2 Hz, 6H).

³¹P NMR (162 MHZ, DMSO) δ −6.83.

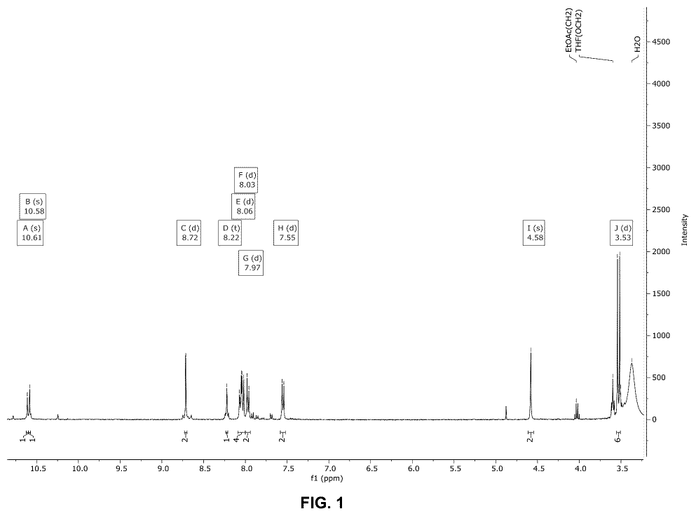

FIG. 1 is a 1H Nuclear magnetic resonance (NMR) spectrum of the azide-containing bis-(acyl phosphate monoester) cross-linker (8).

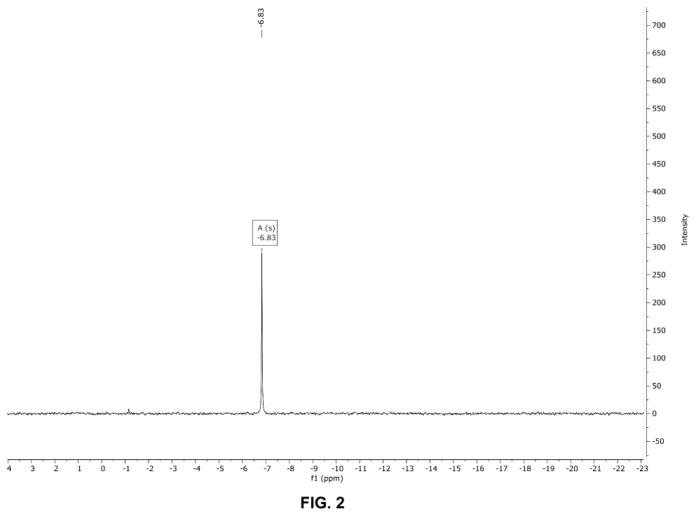

FIG. 2 is a ³¹P NMR spectrum of the azide-containing bis-(acyl phosphate monoester) cross-linker (8).

### Example 2

### Preparation of Cross-Linked Carbonmonoxy Hemoglobin-Azide.

The cross-linked carbonmonoxy hemoglobin-azide was synthesized in accordance with Scheme 3 below.

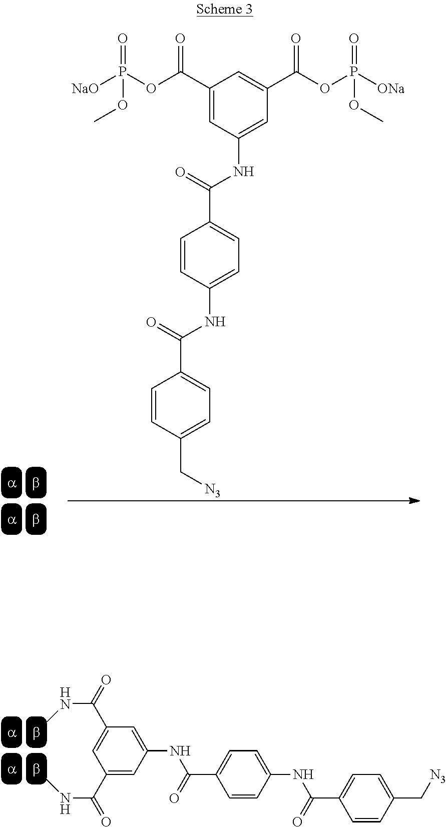

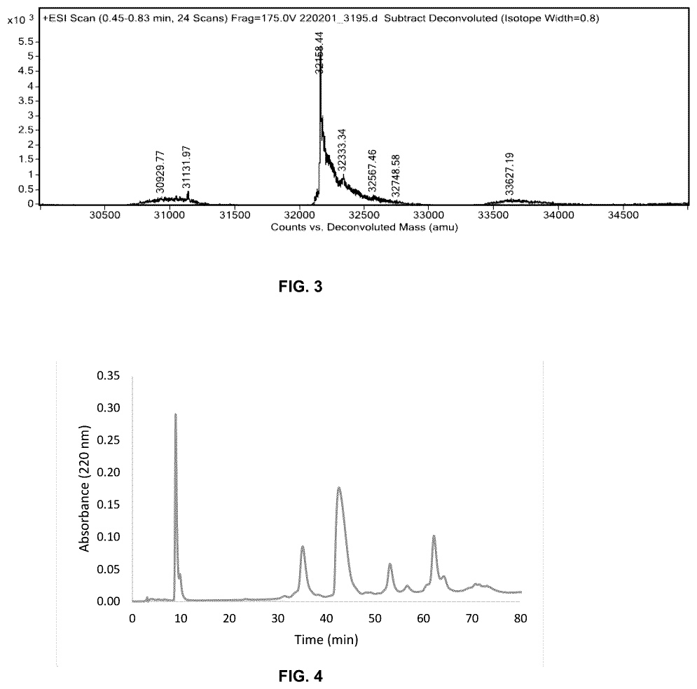

A solution of carbonmonoxy hemoglobin (1.5 mM in 1.0 mL of 0.1 M MOPS, pH 7.2) was placed under a stream of carbon monoxide for 15 minutes at room temperature while stirred. The azide-containing bis-(acyl phosphate monoester) cross-linker (8) (2.1 mg; 2 equivalent) obtained from Example 1 was dissolved 1.0 mL of 0.1 M MOPS, pH 7.2 and added dropwise. After 2 hours, the mixture was passed through a Sephadex™ G-25 column equilibrated with phosphate buffer (0.02 M, pH 7.4). The collected fraction was concentrated by centrifugation through a filter (30 kDa cut-off) and stored under an atmosphere of carbon monoxide at 4° C. The composition of the resulting product was analyzed by HPLC equipped with a 330 Å C-4 Vydac™ reverse-phase column (4.6 mm×250 mm) and a solvent gradient from 20 to 60% acetonitrile in water spiked with 0.1% trifluoroacetic acid. The eluent was monitored at 220 nm. Slight drifts in retention times were observed because solvents were mixed offline. The identities of the peaks were investigated using electrospray ionization mass spectrometry analysis. FIG. 3 is a mass spectrum of the cross-linked carbonmonoxy hemoglobin-azide obtained from Example 2. As can be seen from the mass spectrum, the azide-containing bis-(acyl phosphate monoester) cross-linker (8) selectively formed amide bonds with the β-subunits of the carbonmonoxy-hemoglobin: ((15867.22-1.01)×2)+C₂₃H₁₅N₅O₄ (425.11))=32157.53 g/mol. FIG. 4 is a reverse-phase HPLC trace of the cross-linked carbonmonoxy hemoglobin-azide under dissociating conditions before heat treatment. The peaks are: heme (10 min.); β-subunits (35 min.); a subunits (43 min.); singly modified β-subunits (53 min.); cross-linked β-subunits (62 min.). According to FIG. 4, the azide-containing bis-(acyl phosphate monoester) cross-linker (8) selectively formed amide bonds with the β-subunits of the carbonmonoxy-hemoglobin. The yield for the β-β cross-linking was about 40% according to FIG. 4.

### Example 3

### Heat Treatment of Cross-Linked Carbonmonoxy Hemoglobin-Azide

To purify the cross-linked carbonmonoxy hemoglobin-azide obtained from Example 2, the concentration of the crude product was adjusted to 0.05 mM in phosphate buffer (0.02 M, pH 7.4). The crude product was flushed with carbon monoxide in a seal bottle before placing in 75° C. water bath for 30 minutes while stirred. Then, the mixture was transferred to a falcon tube and centrifuged at 4000 rpm for 20 minutes. The precipitate was discarded, and the supernatant was concentrated by centrifugation through a filter (30 kDa cut-off). The heat-treated sample was stored under an atmosphere of carbon monoxide at 4° C.

The efficiency of the heat treatment was assessed by analysis of the products using HPLC. As noted above, FIG. 4 is a diagram showing a reverse-phase HPLC trace of the cross-linked carbonmonoxy hemoglobin-azide under dissociating conditions before the heat treatment.

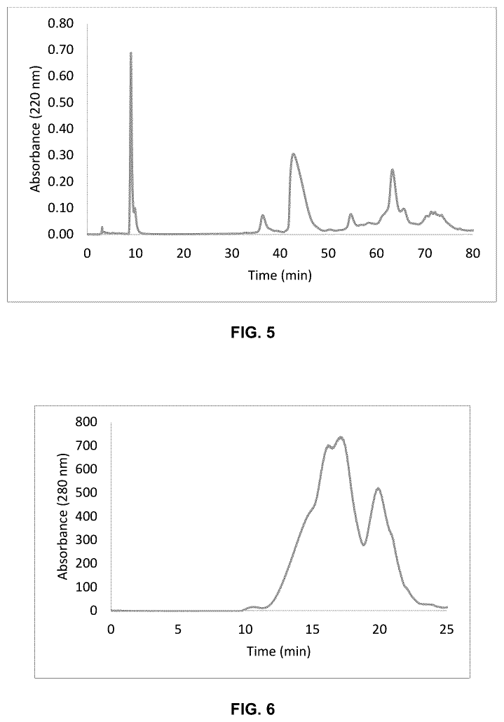

FIG. 5 shows a reverse-phase HPLC trace of the cross-linked carbonmonoxy hemoglobin-azide under dissociating conditions after heat treatment. The peaks include: heme (10 min.); β-subunits (small amount, 36 min.); α-subunits (43 min.); singly modified β-subunits (small amount, 55 min.); cross-linked β-subunits (63 min.).

As can be seen from FIGS. 4 and 5, the cross-linked carbonmonoxy hemoglobin-azide remained stable after heat treatment.

### Example 4

CuAAC of Carbonmonoxy Hemoglobin-Azide with Bis-Alkyne to Form HBOC

Scheme 4 below depicts CuAAC of carbonmonoxy hemoglobin-azide with bis-alkyne to form an HBOC.

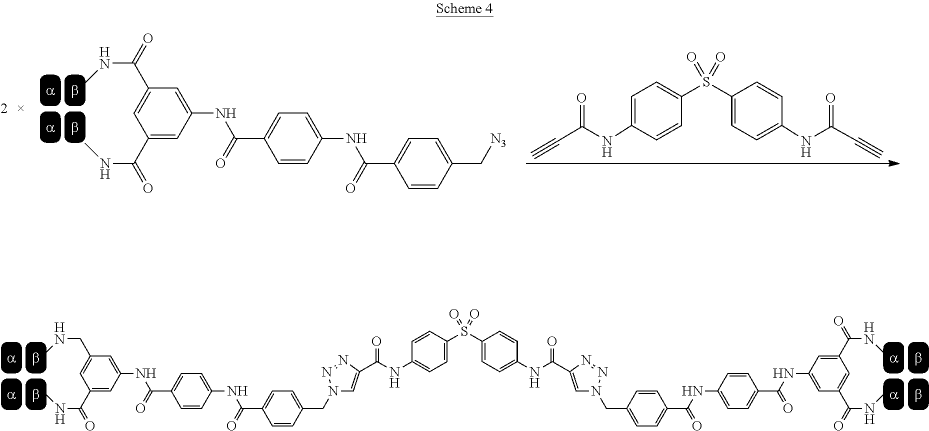

The carbonmonoxy hemoglobin-azide purified by the heat treatment of Example 3 (1 eq., 1 mL of a 0.5 mM stock solution in 0.02 M phosphate buffer, pH 7.4) was passed through a Sephadex™ G-25 column equilibrated with imidazole buffer (0.02 M, pH 7.4). The solution was then transferred to a sealed bottle under an atmosphere of carbon monoxide. Bisalkyne (10 eq., 50 μL, 0.1 M solution in dimethyl sulfoxide), copper sulfate (2.0 equivalents, 50 μL, 0.02 M in water), bath ligand (4 eq., 100 μL, 0.02 M in water), and ascorbic acid (50 eq., 250 μL, 0.1 M in water) were added to the carbonmonoxy hemoglobin-azide solution while stirred. After 4 hours, the mixture was transferred to a falcon tube and centrifuged at 4000 rpm for 20 minutes. The precipitate was discarded, and the supernatant was concentrated by centrifugation through a filter (30 kDa cut-off). Then, the mixture was passed through a Sephadex™ G-25 column equilibrated with phosphate buffer (0.02 M, pH 7.4) to remove excess copper. The product was analyzed by size-exclusion fast protein liquid chromatography (FPLC) (0.05 M phosphate, 0.15 M NaCl, pH 7.4) as shown in FIG. 6.

FIG. 6 shows a size-exclusion FPLC trace of the initially produced HBOC without any purification after the CuAAC reaction. The peaks are: HBOC (128 kDa, 17 min.); unreacted carbonmonoxy hemoglobin tetramers (64 kDa, 20 min.). According to FIG. 6, the overall yield of the HBOC was about 72% with respect to the conversion to the bis-tetramers.

The resulting HBOC was further purified using a size exclusion column (G200 Superdex™ column, 0.05 M phos, 015 M NaCl, pH 7.2).

### Example 5 Oxygen Binding Properties of HBOC

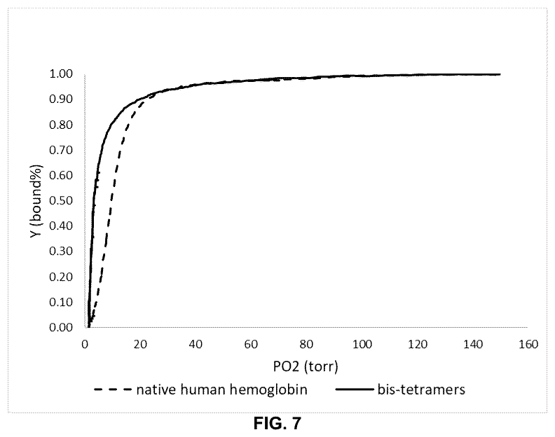

To assess the suitability of the HBOC produced in accordance with Example 4 as a potential oxygen carrier, its oxygen binding properties (P₅₀ and n₅₀) were measured using the method described above. Results were as follows: n₅₀=1.5±0.2; P₅₀=4.0±0.8 torr for HBOC; native hemoglobin n₅₀=2.7±0.2; P₅₀=9.1±0.6 torr. The oxygen binding curves were shown in FIG. 7. The results suggested that the HBOC would likely be able to function as an oxygen carrier in circulation.

It has been observed that the P₅₀ values in HBOCs are generally lower when compared to native hemoglobin, suggesting a lower affinity that provides for a more complete transfer of oxygen from the HBOCs.

While not wishing to be bound by any theory, it is hypothesized that due to the covalent modification by the cross-linkers of the present disclosure, there are likely some restrictions in the conformational change, leading to a lower P₅₀. This would normally promote oxygen to offload at low P₅₀. In other words, a tighter binding means that oxygen can be delivered to more oxygen-depleted areas by circulation or perfusion.

### Example 6 Cross-Linked Carbonmonoxy Hemoglobin-Azide

Example 6 was carried out using 5.0 mL of 0.1 M MOPS, pH 7.2 using an experimental procedure analogous to Example 2.

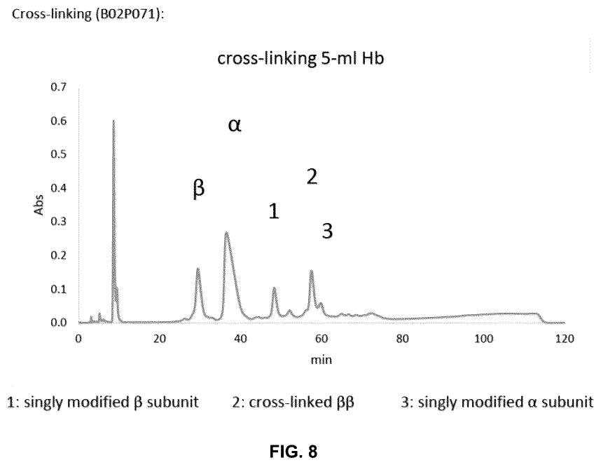

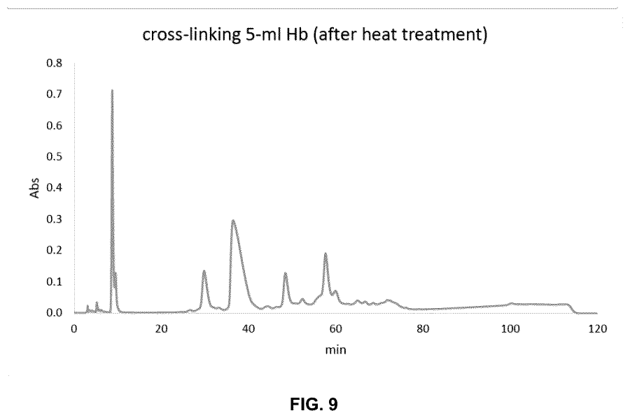

FIG. 8 shows a reverse-phase HPLC trace of the cross-linked carbonmonoxy hemoglobin-azide prepared in accordance with this example under dissociating conditions before heat treatment. FIG. 9 shows a reverse-phase HPLC trace of the cross-linked carbonmonoxy hemoglobin-azide under dissociating conditions after heat treatment.

As can be seen from the above Examples, the use of a novel cross-linker having the azide substituent disclosed herein would lead to substantially β-β cross-linked hemoglobin-azide, which could readily combine with an alkyne having at least two terminal alkyne moieties in the CuAAC process to produce a HBOC in high overall yields, indicative of a scalable production of HBOC.

While the present invention has been described with reference to specific embodiments and examples, it is to be understood that the invention is not limited to the disclosed examples. To the contrary, the present disclosure is intended to cover various modifications and equivalent arrangements. The present disclosure is further intended to cover the application of various alternatives described in respect of one embodiment with other embodiments where it is suitable to do so. Such modifications and arrangements are included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### REFERENCES

The following references are provided as examples of references relating to the present disclosure. The following listing is not intended to comprise a comprehensive list of all relevant art. The entire contents of all references listed in the present specification, including the following documents, are incorporated herein by reference.
1. Lui, F. E.; Kluger, R., Reviving Artificial Blood: Meeting the challenge of dealing with no scavenging by hemoglobin. Chembiochem 2010, 11 (13), 1816-1824.
2. Simoni, J.; Simoni, G.; Moeller, J. F., Intrinsic toxicity of hemoglobin: how to counteract it. Artif. Organs 2009, 33 (2), 100-9.
3. Chang, T. M. S., Hemoglobin-based red blood cell substitutes. Artif. Org. 2004, 28 (9), 789-794.
4. Belcher, D. A.; Cuddington, C. T.; Martindale, E. L.; Pires, I. S.; Palmer, A. F., Controlled polymerization and ultrafiltration increase the consistency of polymerized hemoglobin for use as an oxygen carrier. Bioconj. Chem. 2020, 31 (3), 605-621.
5. Keipert, P.; Minkowitz, J.; Chang, T. M. S., Crosslinked stroma-free polyhemoglobin as a potential blood substitute. Int. J. Artif. Organs 1982, 5 (6), 383-5.
6. Winslow, R. M.; Hess, J.; Macdonald, V. W.; Marini, M. A.; Vandegriff, K. D.; Estep, T., Crosslinked hemoglobin as an oxygen-carrying plasma expander. Clin. Res. 1988, 36 (3), A374-A374.
7. Natanson, C.; Kern, S. J.; Lurie, P.; Banks, S. M.; Wolfe, S. M., Cell-free hemoglobin-based blood substitutes and risk of myocardial infarction and death: a meta-analysis. J. Am. Med. Assoc. 2008, 299 (19), 2304-12.
8. Jahr, J. S.; Guinn, N. R.; Lowery, D. R.; Shore-Lesserson, L.; Shander, A., Blood substitutes and oxygen therapeutics: a review. Anesth. Analg. 2021, 132 (1), 119-129.
9. R. Kluger, A. S. Grant, S. L. Bearne, and M. R. Trachsel “Dicarboxylic Acid bis(methyl phosphates): Anionic Biomimetic Cross-Linking Reagents” J. Org. Chem. 55, 2864-2868 (1990).
10. R. H. Kluger and A. S. Grant “Use of Acyl Phosphate Esters in the Modification of Proteins”, U.S. Pat. No. 5,334,707 (Aug. 2, 1994). 2
11. R. Kluger and J. Wodzinska, “Specifically .beta.-.beta.cross-linked hemoglobins and method of preparation” U.S. Pat. No. 5,250,665, Oct. 5, 1993
12. Frostell, C. G.; Blomqvist, H.; Hedenstierna, G.; Lundberg, J.; Zapol, W. M., inhaled nitric-oxide selectively reverses human hypoxic pulmonary vasoconstriction without causing systemic vasodilation. Anesthesiology 1993, 78 (3), 427-435.
13. Akopov, S. E., Nitric oxide (EDRF) enhances the vasorelaxing effect of dihydropyridine calcium antagonists in isolated human middle cerebral arteries. Methods Find. Exp. Clin. Pharmacol. 1996, 18 (2), 101-104.
14. Alayash, A. I., Hemoglobin-based blood substitutes: oxygen carriers, pressor agents, or oxidants? Nature Biotechnology 1999, 17 (6), 545-549.
15. Vogel, R., Listening to the endothelium-A story of signal and noise. J. Am. Coll. Cardiol. 2008, 51 (20), 1965-1966.
16. Lei, C.; Yu, B.; Shahid, M.; Beloiartsev, A.; Bloch, K. D.; Zapol, W. M., Inhaled nitric oxide attenuates the adverse effects of transfusing stored syngeneic erythrocytes in mice with endothelial dysfunction after hemorrhagic shock. Anesthesiology 2012, 117 (6),
17. Fleming, I., Molecular mechanisms underlying the activation of eNOS. Pflugers Archiv-Eur. J. Physiol. 2010, 459 (6), 793-806.
18.Sloan, E. P.; Koenigsberg, M.; Gens, D.; Cipolle, M.; Runge, J.; Mallory, M. N.; Rodman, G.; Shoc, D. T. H., Diaspirin cross-linked hemoglobin (DCLHb) in the treatment of severe traumatic hemorrhagic shock-A randomized controlled efficacy trial. JAMA 1999, 282 (19), 1857-1864.
19. Vandegriff, K. D.; Malavalli, A.; Wooldridge, J.; Lohman, J.; Winslow, R. M., MP4, a new nonvasoactive PEG-Hb conjugate. Transfusion 2003, 43 (4), 509-516.
20. Yang, Y.; Kluger, R., Efficient CuAAC click formation of functional hemoglobin 27 bis-tetramers. Chem. Commun. 2010, 46 (40), 7557-7559.
21. Wang, A.; Singh, S.; Yu, B. L.; Bloch, D. B.; Zapol, W. M.; Kluger, R., Cross-linked hemoglobin bis-tetramers from bioorthogonal coupling do not induce vasoconstriction in 2 the circulation. Transfusion 2019, 59 (1), 359-370.
22. Lui, F. E.; Yu, B. L.; Baron, D. M.; Lei, C.; Zapol, W. M.; Kluger, R., Hemodynamic responses to a hemoglobin bis-tetramer and its polyethylene glycol conjugate. Transfusion 2012, 52 (5), 974-982.
23. Foot, J. S.; Lui, F. E.; Kluger, R., Hemoglobin bis-tetramers via cooperative azide-alkyne coupling Chem. Commun. 2009, 7315-7317.
24. Wang, A.; Kluger, R., Increasing efficiency in protein-protein coupling: subunit-directed acetylation and phase-directed CuAAC (“click coupling”) in the formation of hemoglobin bis-tetramers. Biochemistry 2014, 53 (43), 6793-9.

What is claimed is:

1. An azide-containing cross-linker, wherein the cross-linker is a compound of Formula I, II, III or IV 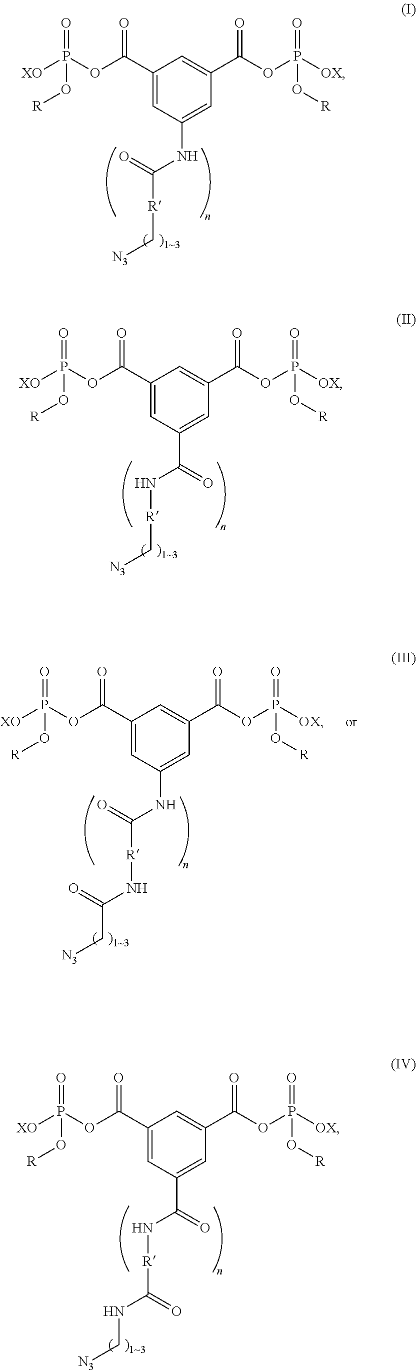
wherein R is an alkyl;
X is an alkali ion;
n is 1 to 5; and
R′ is a para- or meta-substitutable benzene or benzene isomer, a para- or meta-substitutable heterocyclic six-membered aromatic ring, a five membered aromatic ring, an indole ring, an indole derivative, a naphthalene or a naphthalene derivative, wherein each of the para- or meta-substitutable benzene, the para- or meta-substitutable benzene isomer, the para- or meta-substitutable heterocyclic six-membered aromatic ring, the five membered aromatic ring, the indole ring, the indole derivative, the naphthalene and the naphthalene derivative is optionally substituted.

2. The azide-containing cross-linker of claim 1, wherein R is methyl or ethyl; and X is Li or Na.

3. The azide-containing cross-linker of claim 1, wherein
R′ is mono, bis or tris substituted with 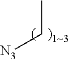
on at least one aromatic ring thereof; or

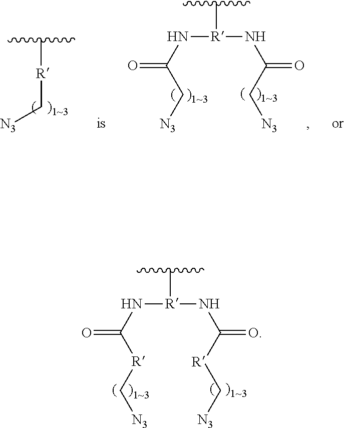

4. The azide-containing cross-linker of claim 1, wherein
the heterocyclic six-membered aromatic ring is 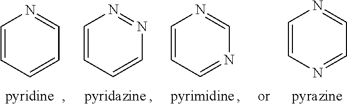
or isomers thereof;
the five-membered aromatic ring is 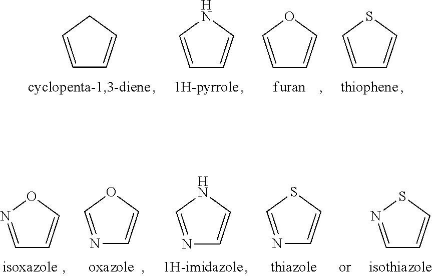
or isomers thereof;
the indole ring derivative is 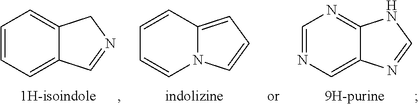
the naphthalene derivative is 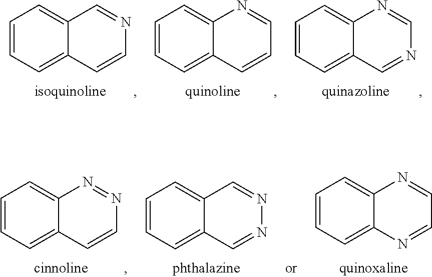
or isomers thereof; and
each of the para- or meta-substitutable benzene, the para- or meta-substitutable benzene isomer, the para- or meta-substitutable heterocyclic six-membered aromatic ring, the five membered aromatic ring, the indole ring, the indole derivative, the naphthalene, the naphthalene derivative and isomers thereof is optionally substituted with methyl, ethyl, allyl, propyl, isopropyl, fluoro, chloro, bromo, iodo, hydroxyl, methoxy, ethyoxy, isopropyl, carboxyl, acetyl, cyano, or a nitro group on the at least one aromatic ring.

5. An azide-containing cross-linker of Formula V, 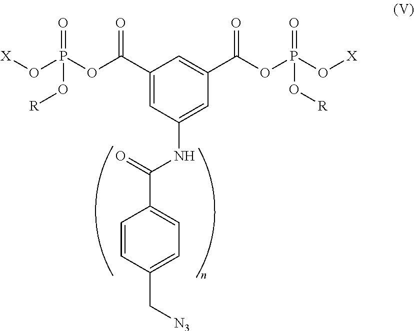
wherein R is an alkyl;
X is an alkali ion; and
n is 1 to 5.

6. The azide-containing cross-linker of claim 5, wherein R is methyl or ethyl, X is Li or Na and n is 2 to 5.

7. A method for preparing the azide-containing cross-linker of claim 1, wherein the method is one of Schemes 1a to 1e below depending on the azide-containing cross-linker to be prepared: 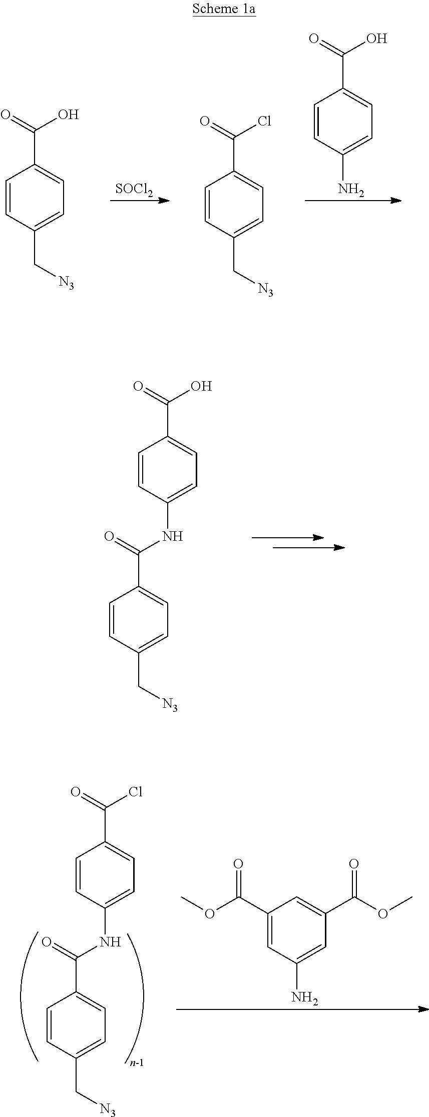 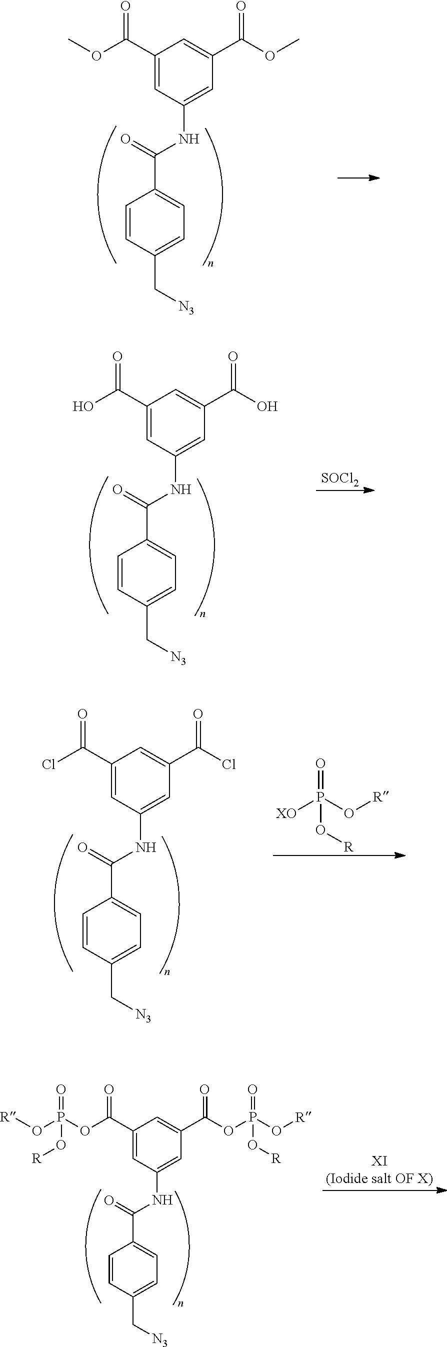 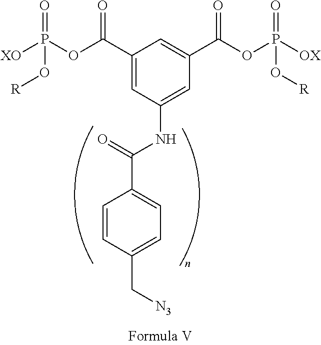 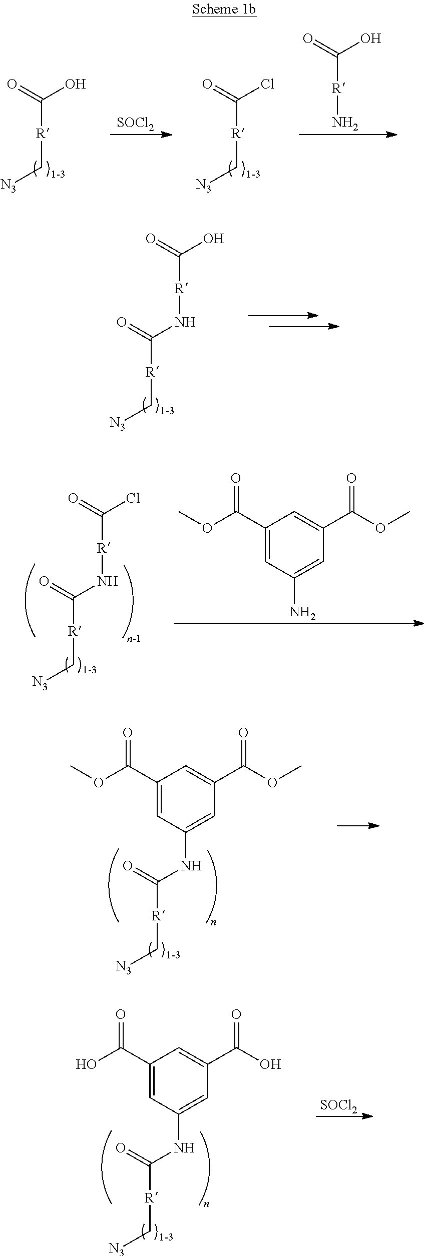 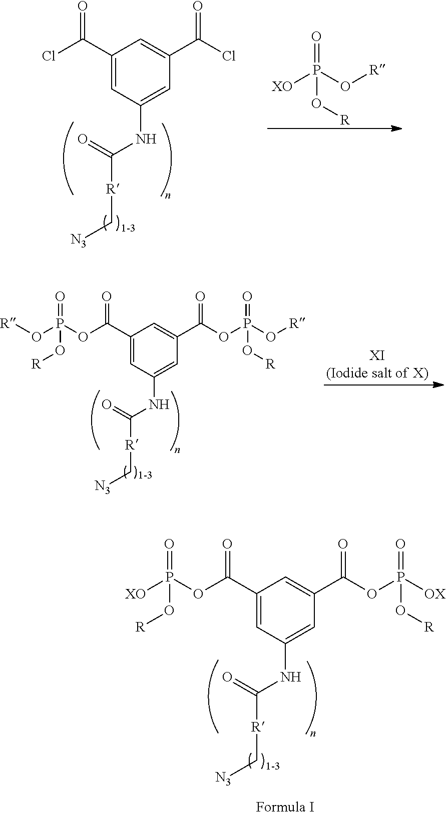 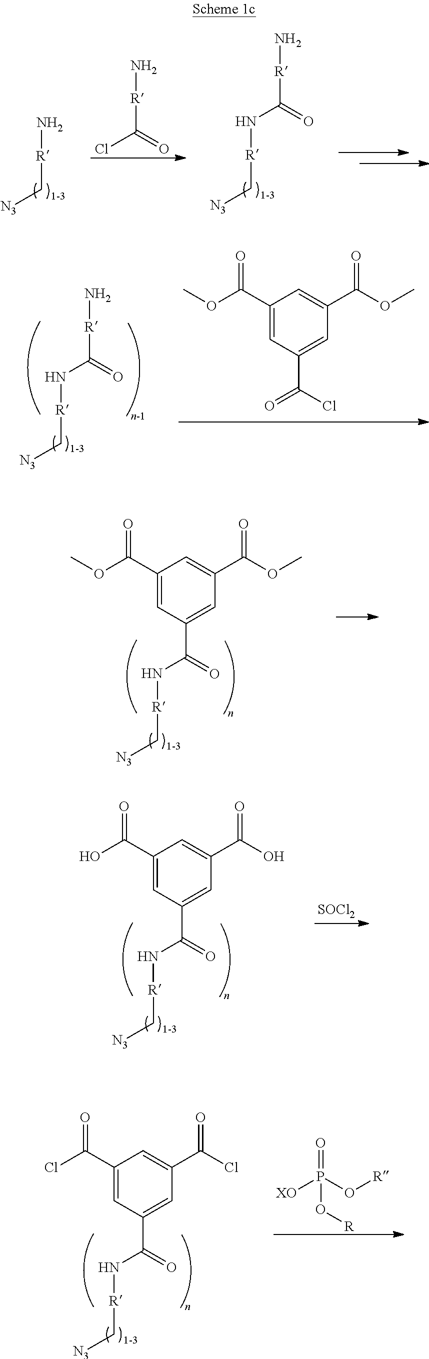 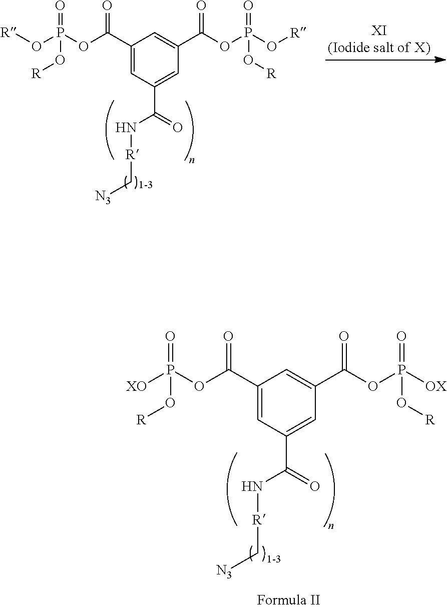 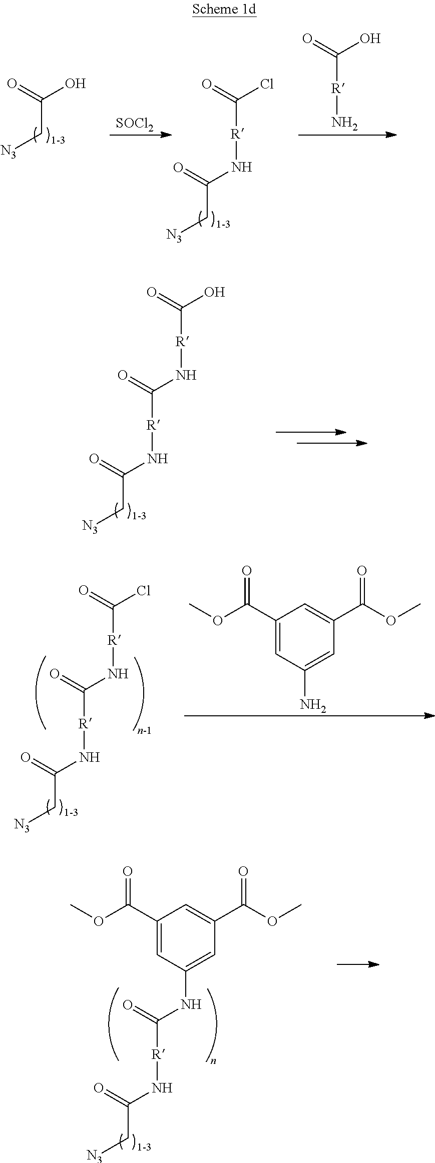 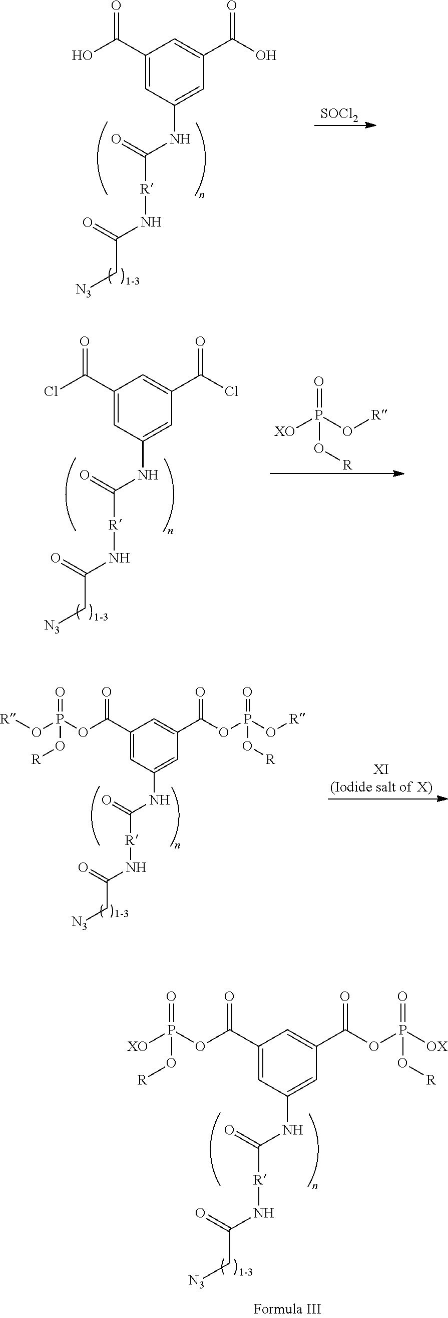 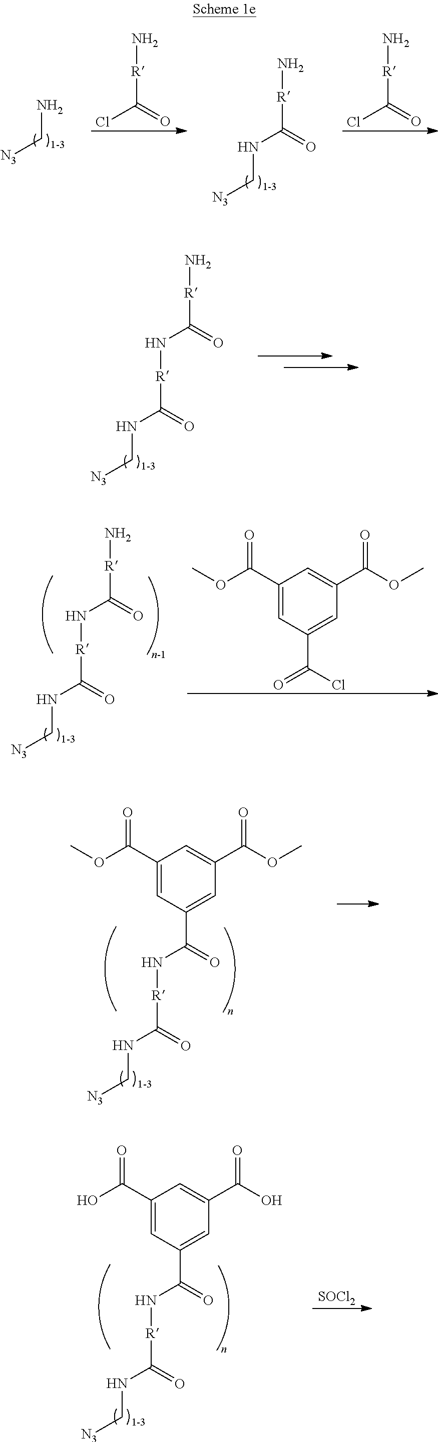 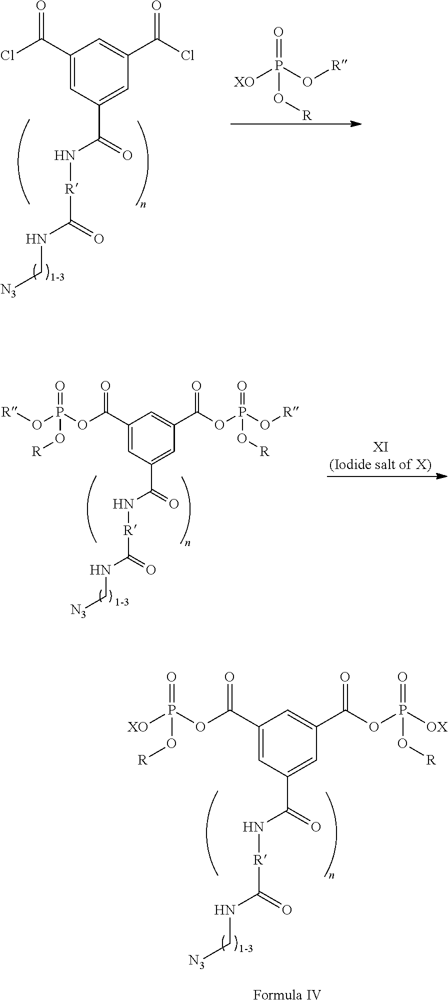
wherein in each of Schemes 1a to 1e, R″ is the same as or different from R and R″ is methyl or ethyl; and X is an alkali ion; and the double arrows represent repeating steps 1 and 2 to extend the azide-containing moiety to a specified hydrocarbon chain length where n=1 to 5.

8. A cross-linked hemoglobin protein comprising a hemoglobin protein cross-linked by the azide-containing cross-linker of claim 1, wherein the hemoglobin protein comprises two alpha and two beta subunits.

9. The cross-linked hemoglobin protein of claim 8, wherein each of the beta subunits of the hemoglobin protein is covalently linked via an amide bond to the azide-containing cross-linker.

10. The cross-linked hemoglobin protein of claim 8, wherein the cross-linked hemoglobin protein is represented by Formula VI

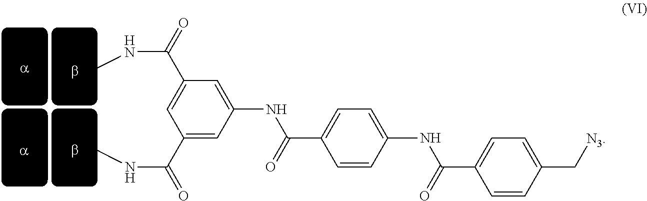 [SMILES: CNC(=O)c1cc(NC(=O)c2ccc(NC(=O)c3ccc(CN=[N+]=[N-])cc3)cc2)cc(C(=O)NC)c1]

11. The cross-linked hemoglobin of claim 8, wherein the hemoglobin protein is deoxyhemoglobin, carbonmonoxyhemogloblin or oxyhemoglobin.

12. A hemoglobin-based oxygen carrier (HBOC), comprising a pair of the cross-linked hemoglobin of claim 8 coupled by a copper catalyzed azide-alkyne cycloaddition (CuAAC).

13. The HBOC of claim 12, wherein the alkyne is a bis-alkyne, tris-alkyne or tetrakis-alkyne.

14. The HBOC of claim 13, wherein the bis-alkyne is

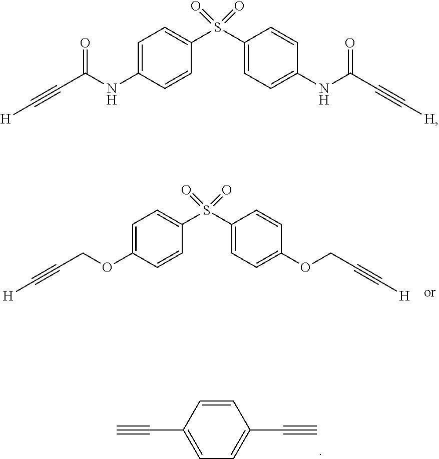

15. The HBOC of claim 13, wherein the tris-alkyne is

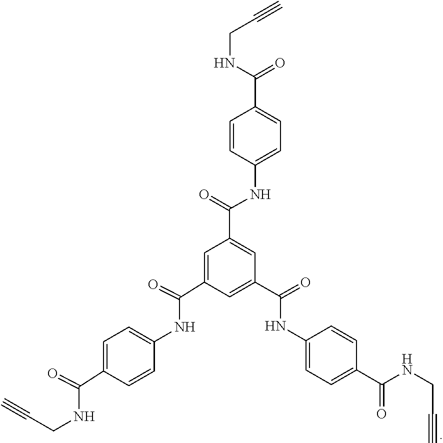 [SMILES: C#CCNC(=O)c1ccc(NC(=O)c2cc(C(=O)Nc3ccc(C(=O)NCC#C)cc3)cc(C(=O)Nc3ccc(C(=O)NCC#C)cc3)c2)cc1]

16. The HBOC of claim 13, wherein the tetrakis-alkyne is

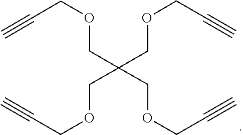 [SMILES: C#CCOCC(COCC#C)(COCC#C)COCC#C]

17. The HBOC of claim 12, wherein the HBOC is represented by Formula VII

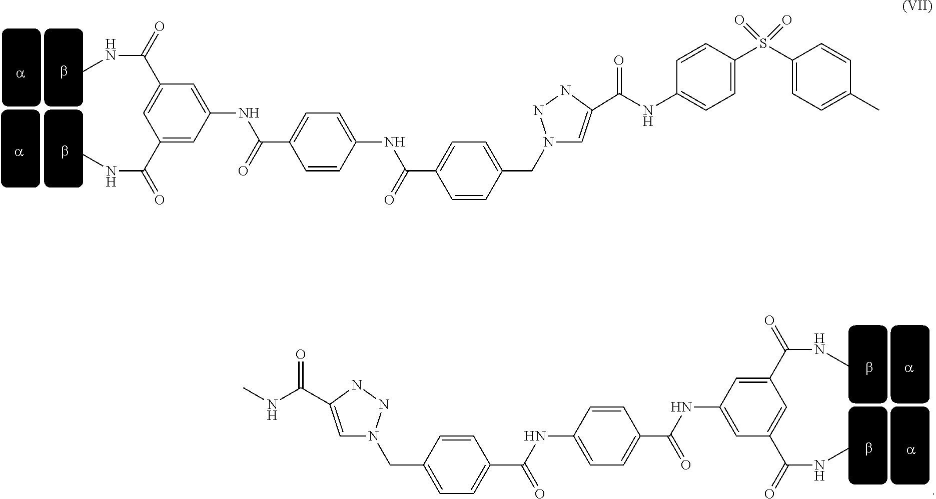

18. A composition for use to increase oxygen transport, comprising the HBOC of claim 12 and a suitable carrier.

19. The composition of claim 18 for use in perfusion or transfusion.
